(19) United States
(12) Patent Application Publication
(10) Pub. No.: US 2019/0247010 A1
(43) Pub. Date: Aug. 15, 2019

(54) Infrasound biosensor system and method

(71) Applicant: Anna Barnacka, Cambridge (US)

(72) Inventor: Anna Barnacka, Cambridge (US)

(21) Appl. No.: 16/274,873

(22) Filed: Feb. 13, 2019

Related U.S. Application Data
(60) Provisional application No. 62/629,961, filed on Feb. 13, 2018.

(51) Int. Cl. A61B 8/02; A61B 8/06; A61B 5/00

(52) U.S. Cl. CPC A61B 8/02; A61B 8/065; A61B 8/5223; A61B 5/6803; A61B 5/6814

(57) ABSTRACT

A portable infrasonic body activity monitoring system including a headset and portable device. The headset is equipped with a set of microphones and auxiliary sensors including thermometers, gyroscopes, accelerometers. The set of microphones detect acoustic signals in the audible frequency bandwidth and in the infrasonic bandwidth. The headset can have a form of earphones or headphones. Monitored infrasound is a result of blood flow and oscillations related to brain activity, and results in measuring a range of parameters including heart rate, breathing rate, etc. The brain and body activity can be monitored through software running on the mobile device. The mobile device can be wearable. The invention can be used for biofeedback.

### RELATED APPLICATIONS

This application claims the benefit under 35 USC 119(e) of U.S. Provisional Application No. 62/629,961, filed on Feb. 13, 2018, which is incorporated herein by reference in its entirety.

### BACKGROUND OF THE INVENTION

Physical wellbeing is vital to human health and happiness. Body activity monitoring is crucial to our understanding of health and body function and their response to external stimuli.

Monitoring personal health and body function is currently performed by a plethora of separate medical devices and discrete monitoring devices. Heart rate, body temperature, respiration, cardiac performance and blood pressure are measured by separate devices. The medical versions of current monitoring devices set a standard for measurement accuracy, but they sacrifice availability, cost and convenience. Consumer versions of body function monitoring devices are generally more convenient and inexpensive, but they are typically incomplete and in many cases inaccurate.

### SUMMARY OF THE INVENTION

The invention of acoustic biosensor technology combines medical device precision over a full range of biometric data with the convenience, low cost, and precision needed to make health and wellness monitoring widely available and effective.

Accordingly, there is a need for a possibly portable body activity monitoring device that can be discreet, accessible, easy to use, and cost-efficient. Such a device could allow for real-time monitoring of body activity over an extended period and a broad range of situations, for example.

The present invention can be implemented as an accessible and easy to use body activity monitoring system, or biosensor system, including a head-mounted transducer system and a processing system. The head-mounted transducer system is equipped with one or more acoustic transducers, e.g., microphones or other sensors capable of detecting acoustic signals from the body. The acoustic transducers detecting acoustic signals in the infrasonic band and/or audible frequency band. The head-mounted transducer system also preferably includes auxiliary sensors including thermometers, accelerometers, gyroscopes, etc. The head-mounted transducer system can take the form of a headset, earbuds, earphones and/or headphones. In many cases, the acoustic transducers are installed outside, at the entrance, and/or inside the ear canal of the user. The wearable transducer system can be integrated discretely with fully functional audio earbuds or earphones, permitting the monitoring functions to collect biometric data while the user listens to music, makes phone calls, or generally goes about their normal life activities.

Generally, monitored biological acoustic signals are the result of blood flow and other vibrations related to body activity. The head-mounted transducer system provides an output data stream of detected acoustic signals and other data generated by the auxiliary sensors to the processing system such as a mobile computing device, such as for example, a smartphone or smartwatch or other carried or wearable mobile computing device and/or server systems connected the transducer system and/or the mobile computing device.

The acoustic transducers typically include at least one microphone. More microphones can be added. For example, microphones can be embodied in earphones that detect air pressure variations of sound waves in the user's ear canals and convert the variations into electrical signals. In addition, or in the alternative, other sensors can be used to detect the biological acoustic signals such as displacement sensors, contact acoustic sensors, strain sensors, to list a few examples.

The head-mounted transducer system can additionally have speakers that generate sound in the audible frequency range, but can also generating sound in the infrasonic range. The innovation allows for monitoring for example vital signs including heart and breathing rates, and temperature, and also blood pressure and circulation. Other microphones can be added to collect and record background noise. One of the goals of background microphones can be to help discriminate between acoustic signals originating from the user's brain and body from external noise. In addition, the background microphones can monitor the external audible and infrasound noise and can help to recognize its origin. Thus, the user might check for the presence of infrasound noise in the user's environment.

Body activity can be monitored and characterized through software running on the processing system and/or a remote processing system. The invention can for example be used to monitor body activity during meditation, exercising, sleep, etc. It can be used to establish the best level of brain and body states and to assess the influence of the environment, exercise, the effect of everyday activities on the performance, and can be used for biofeedback, among other things.

In general, according to one aspect, the invention features a biosensor system, comprising an acoustic sensor for detecting acoustic signals from a user via an ear canal and a processing system for analyzing the acoustic signals detected by the acoustic sensor.

In embodiments, the system acoustic signals include infrasounds and/or audible sounds. Moreover, the system preferably further has auxiliary sensors for detecting movement of the user, for example. In additional, an auxiliary sensor for detecting a body temperature of the user is helpful.

In many cases, the acoustic sensor is incorporated into a headset. The headset might include one or more earbuds. Additionally some means for occluding the ear canal of the user is useful to improve an efficiency of the detection of the acoustic signals. The occluding means could include an earbud cover.

Preferably, there are acoustic sensors in both ear canals of the user and the processing system uses the signals from both sensors to increase an accuracy of a characterization of bodily process such as cardiac activity and/or respiration.

Usually, the processing system analyzes the acoustic signals to analyze a cardiac cycle and/or respiratory cycle of the user.

In general, according to another aspect, the invention features a method for monitoring a user with a biosensor system. Here, the method comprises detecting acoustic signals from a user via an ear canal using an acoustic sensor and analyzing the acoustic signals detected by the acoustic sensor to monitor the user.

In general, according to another aspect, the invention features an earbud-style head-mounted transducer system. It comprises an ear canal extension that projects into an ear canal of a user and an acoustic sensor in the ear canal extension for detecting acoustic signals from the user.

In general, according to another aspect, the invention features a user device executing an app providing a user interface for a biosensor system on a touchscreen display of the user device. This biosensor system analyzes infrasonic signals from a user to assess a physical state of the user. Preferably, the user interface presents a display that analogizes the state of the user to weather and/or presents the plots of infrasonic signals and/or a calendar screen for accessing past vital state summaries based on the infrasonic signals.

In general, according to another aspect, the invention features a biosensor system and/or its method of operation, comprising one or more acoustic sensors for detecting acoustic signals including infrasonic signals from a user and a processing system for analyzing the acoustic signals to facilitate one or more of the following: environmental noise monitoring, blood pressure monitoring, blood circulation assessment, brain activity monitoring, circadian rhythm monitoring, characterization of and/or assistance in the remediation of disorders including obesity, mental health, jet lag, and other health problems, meditation, sleep monitoring, fertility monitoring, and/or menstrual cycle monitoring.

In general, according to yet another aspect, the invention biosensor system and/or method of its operation, comprising an acoustic sensor for detecting acoustic signals from a user, a background acoustic sensor for detecting acoustic signals from an environment of the user, and a processing system for analyzing the acoustic signals from the user and from the environment.

In examples, the biosensor system and method might characterize audible sound and/or infrasound in the environment using the background acoustic sensor. In addition, the biosensor system and method will often reduce noise in detected acoustic signals from the user by reference to the detected acoustic signals from the environment and/or information from auxiliary sensors.

The above and other features of the invention including various novel details of construction and combinations of parts; and other advantages; will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings; reference characters refer to the same parts throughout the different views. The drawings are not necessarily to scale; emphasis has instead been placed upon illustrating the principles of the invention. Of the drawings:

FIG. 1 is a schematic diagram showing a head-mounted transducer system of a biosensor system, including a user device, and cloud server system, according to the present invention;

FIG. 2 is a human audiogram range diagram showing the ranges of different human origination sounds are depicted with signal of interest corresponding to cardiac activity, detectable below 10 Hz;

FIG. 3 are plots of amplitude in arbitrary units as a function of time in seconds showing raw data recorded with microphone located inside right ear canals (dotted line) and left ear canal (solid line);

FIG. 4A is a plot of a single waveform corresponding to a cardiac cycle with an amplitude in arbitrary units as a function of time in seconds recorded with a microphone located inside the ear canal, note: the large amplitude signal around 0.5 seconds corresponds to the ventricular contraction. FIG. 4B shows multiple waveforms of cardiac cycles with an amplitude in arbitrary units as a function of time in seconds showing infrasound activity over 30 seconds recorded with a microphone located inside the ear canal;

FIGS. 5A and 5B are power spectra of data presented in FIG. 4B, FIG. 5A shows magnitude in decibels as a function of frequency, log scale. FIG. 5B shows an amplitude in arbitrary units and linear scale. Dashed lines in FIG. 4A indicate ranges corresponding to different brain waves detectable with EEG. The prominent peaks in FIG. 5B below 10 Hz correspond mostly to the cardiac cycle;

FIG. 6 is a schematic diagram showing earbud-stele head-mounted transducer system of the present invention;

FIG. 7 is a schematic diagram showing the printed circuit board of the earbud-style head-mounted transducer system;

FIG. 8 is a schematic diagram showing a control module for the head-mounted transducer system;

FIG. 9 is a circuit diagram of each of the left and right analog channels of the control module;

FIG. 10 depicts an exploded view of an exemplary earphone/earbud style transducer system according to an embodiment of the invention;

FIG. 11 is a block diagram illustrating the operation of the biosensor system 50;

FIG. 12 is a flowchart for signal processing of biosensor data according to an embodiment of the invention;

FIGS. 13A, 13B, 13C, and 13D are plots over time showing phases of data analysis used to extract cardiac waveform and obtain biophysical metrics such as heart rate, heart rate variability, respiratory sinus arrhythmias, breathing rate;

FIG. 14 shows data assessment flow and presents data analysis flow;

FIG. 15 is a schematic diagram showing a network 1200 supporting communications to and from biosensor systems 50 for various users;

FIGS. 16A-16D shows four exemplary screenshots of the user interface of an app executing on the user device 106.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present system makes use of acoustic signals generated by the blood flow, muscles, mechanical motion, and neural activity of the user. It employs acoustic transducers, e.g., microphones, and/or other sensors, embedded into a head-mounted transducer system, such as, for example a headset or earphones or headphones, and possibly elsewhere to characterize a user's physiological activity and their audible and infrasonic environment. The acoustic transducers, such as one or an array of microphones, detects sound in the infrasonic and audible frequency ranges, typically from the user's ear canal. The other, auxiliary, sensors may include but are not limited to thermometers; accelerometers, gyroscopes, etc.

The present system enables physiological activity recording, storage, analysis, and/or biofeedback of the user. It can operate as part of an application executing on the local processing system and can further include remote processing system(s) such as a web-based computer server system for more extensive storage and analysis. The present system provides information on a user's physiological activity including but not limited to heart rate and its characteristics, breathing and its characteristics, body temperature, the brain's blood flow including but not limited to circulation and pressure, neuronal oscillations, user motion, etc.

Certain embodiments of the invention include one or more background or reference microphones—generally placed on one or both earphones—for recording sound, in particular infrasound but typically also audible sound, originating from the user's environment. These signals are intended to be used to enable the system to distinguish and discriminate between sounds originating from the user's body from the user's environment and also characterize the environment. The reference microphones can further be used to monitor the level and origin of audible and infrasound in the environment.

The following description provides exemplary embodiments only, and is not intended to limit the scope, applicability or configuration of the disclosure. Rather, the following description of the exemplary embodiment(s) will enable those skilled in the art to implement an exemplary embodiment. It being understood that various changes may be made in the function and arrangement of elements without departing from the spirit and scope as set forth in the appended claims.

Various embodiments of the invention may include assemblies which are interfaced wirelessly and/or via wired interfaces to an associated electronics device providing at least one of: pre-processing, processing, and/or analysis of the data. The head-mounted transducer system with its embedded sensors may be wirelessly connected and/or wired to the processing system, which is implemented in an ancillary, usually a commodity, portable, electronic device, to provide recording, preprocessing, processing, and analysis of the data discretely as well as supporting other functions including, but not limited to, Internet access, data storage, sensor integration with other biometric data, user calibration data storage, and user personal data storage.

The processing system of the biosensor monitoring system as referred herein and throughout this disclosure, can be implemented a number of ways. It should generally have wireless and/or wired communication interfaces and have some type of energy storage unit such as a battery for power and/or have a fixed wired interface to obtain power. Wireless power transfer is another option. Examples include (but are not limited to) cellular telephones, smartphones, personal digital assistants, portable computers, pagers, portable multimedia players, portable gaming consoles, stationary multimedia players, laptop computers, computer services, tablet computers, electronic readers, smartwatches (e.g., iWatch), personal computers, electronic kiosks, stationary gaming consoles, digital set-top boxes, and Internet-enabled applications, GPS enabled smartphones running the Android or IOS operating systems, GPS units, tracking units, portable electronic devices built for this specific purposes, personal digital assistant, NEN players, iPads, cameras, handheld devices, pagers. The processing system may also be wearable.

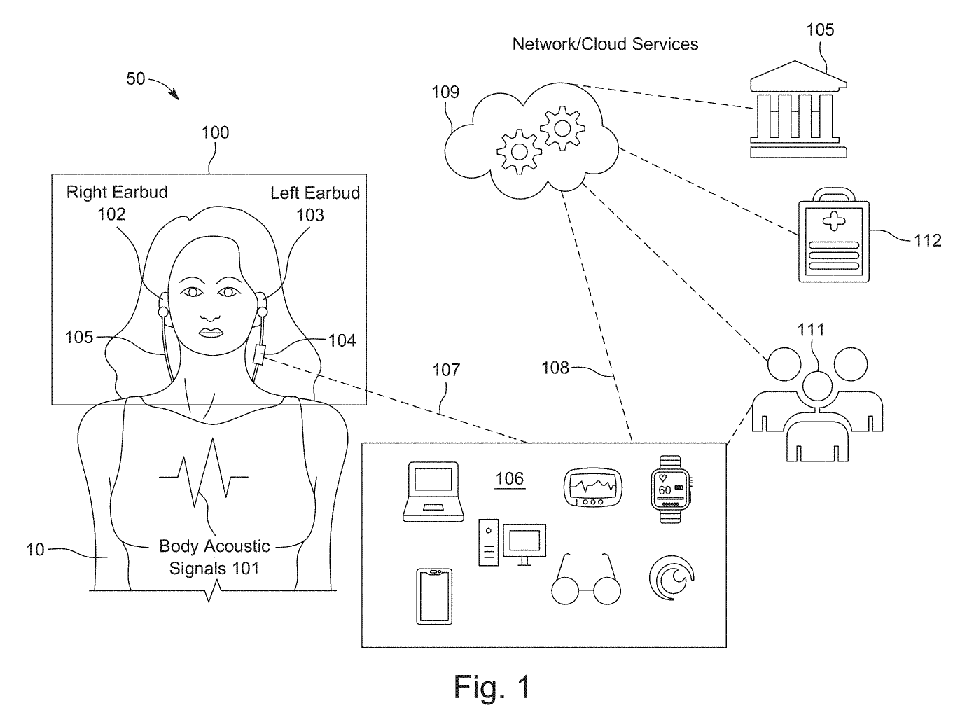

FIG. 1 depicts an example of a biosensor system 50 that has been constructed according to the principles of the present invention.

In more detail, a user 10 wears a head-mounted transducer system 100 in the form of right and left earbuds 102, 103, in the case of the illustrated embodiment. The right and left earbuds 102, 103 mount at the entrance or inside the user's two ear canals. The housings of the earbuds may be shaped and formed from a flexible, soft material or materials. The earphones can be offered in range of colors, shapes, and sizes. Sensors embedded into right and left earbuds 102, 103 or headphones will help promote consumer/market acceptance, i.e. widespread general-purpose use.

The right and left earbuds 102, 103 are connected via a tether or earbud connection 105. A control module 104 is supported on this tether 105.

It should be noted that this is just one potential embodiment in which the head-mounted transducer system 100 is implemented as a pair of tethered earbuds.

Infrasounds

Biological acoustic signals 101 are generated internally in the body by for example breathing, heartbeat, coughing, muscle movement, swallowing, chewing, body motion, sneezing, blood flow, etc. Audible and infrasonic sounds can be also generated by external sources, such as air conditioning systems, vehicle interiors, various industrial processes, etc.

Acoustic signals 101 represent fluctuating pressure changes superimposed on the normal ambient pressure, and can be defined by their spectral frequency components. Sounds with frequencies ranging from 20 Hz to 20 kHz represent those typically heard by humans and are the designated as falling within the audible range. Sounds with frequencies below the audible range are termed infrasonic. The boundary between the two is somewhat arbitrary and there is no physical distinction between infrasound and sounds in the audible range other than their frequency and the efficiency with which the modality by which they are sensed by people. Moreover, infrasound often becomes perceptible to humans if the sound pressure level is high enough by the sense of touch.

The level of a sound is normally defined in terms of the magnitude of the pressure changes it represents, which can be measured and which does not depend on the frequency of the sound. The biologically-originating sound inside the ear canal is mostly in infrasound range. Occluding an ear canal with for example an earbud as proposed in this inventions, amplifies the body infrasound in the ear canal and facilitate the signal detection.

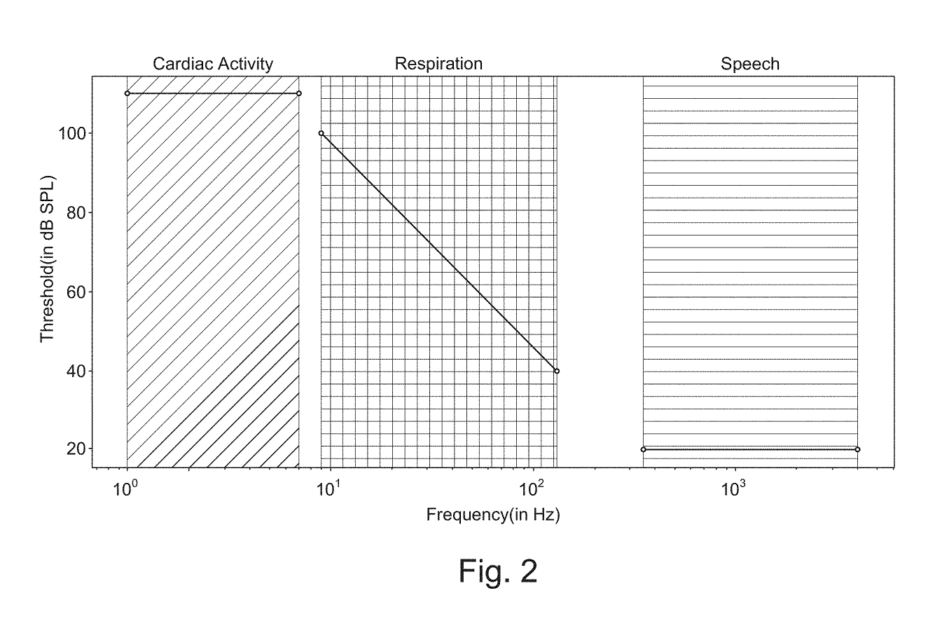

FIG. 2 shows frequency ranges corresponding to cardiac activity, respiration, and speech. Accordingly, it is difficult to detect internal body sound below 10 Hz with standard microphone circuits with the typical amount of noise that may arise from multiple sources, including but not limited to the circuit itself and environmental sounds. The largest circuit contribution to the noise is the voltage noise. Accordingly, some embodiments of the invention reduce the noise using array of microphones and by summing the signal. In this way, the real signal that is correlated sums up, while, the circuit noise, which has characteristic of white noise is reduced.

Other sources of the circuit noise include, but are not limited to:

Resistance: in principle, resistors have a tolerance of the order of 1%. As a result, the voltage drop across resistors can be off by 1% or higher. This resistor characteristic can also change over the resistor lifetime. Such change does not introduce errors on short time scales. However, it introduces possible offsets to a circuit's baseline voltage. A typical resistor's current noise is in the range from 0.2 to 0.8 (mu V/V).

Capacitance: capacitors can have tolerances of the order of 5%. As a result, the voltage drop across them can be off by 5% or more, with typical values reaching even 20%. This can result in an overall drop to the voltage (and therefore signal) in the circuit, however rapid changes are rare. Their capacitance can also degrade with very cold and very hot temperatures.

Microphones: A typical microphone noise level is of the order of 1-2%, and is dominated by electrical (I/O noise.

Operational amplifiers: For low microphone impedances the electrical (known also as a voltage or 1/f) noise dominates. In general, smaller size microphones have a higher impedance. In such systems equipped with high impedance, the current noise can start dominating. In addition, the operational amplifier can be saturated if the input signal is too loud, which can lead to a period of distorted signals. In the low impedance systems, the microphone's noise is the dominating source of noise (not the operational amplifier).

Voltage breakdown: in principle, all components can start to degrade if too high of a voltage is applied. A system with low voltage components is a one of solutions to avoid the voltage breakdown.

Bio-Infrasound Signal

Returning back to FIG. 1, typically the user 10 is provided with or uses a processing system 106, such as for example, a smartphone, a tablet computers (e.g., iPad brand computer), a smart watch (e.g., iWatch brand smartwatch), laptop computer or other portable computing device, which has a connection via the wide area cellular data network or a, WiFi network, or other wireless connection such as Bluetooth to other phones, the Internet, or other wireless networks for data transmission to possible a web-based cloud computer server system 109 that functions as part of the processing system.

The head-mounted transducer system 100 captures body and environmental acoustic signals by way of acoustic sensors such as microphones, which respond to vibrations from sounds.

In some examples, the right and left earbuds 102, 103 connect to an intervening controller module 104 that maintains a wireless connection 107 to the processing system or user device 106 and/or the server system 109. In turn, the user device 106 maintains typically a wireless connection 108 such as via a cellular network or other wideband network or Wi-Fi networks to the cloud computer server system 109. From either system, information can be obtained from medical institutions 105, medical records repositories 112, possibly other user devices 111.

It should be noted that the controller module 104 is not discrete from the earbuds or other headset, in some implementations. It might be integrated into one or both of the earbuds, for example.

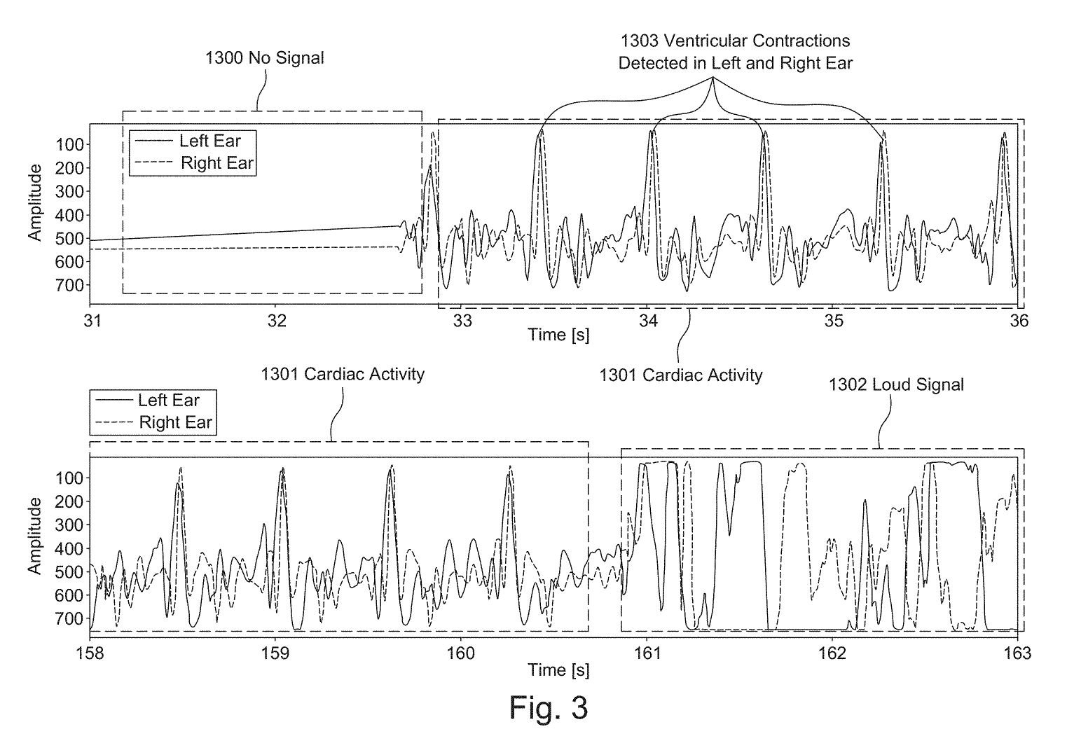

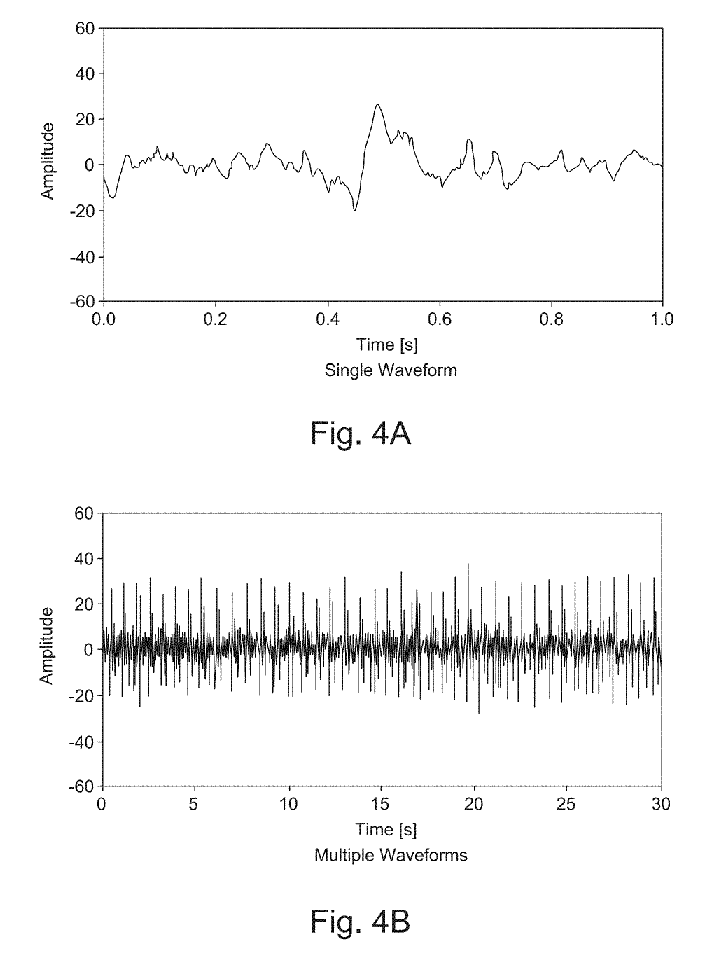

FIGS. 3 and 4A, 4B show exemplary body physiological activity recorded with a microphone located inside the ear canal.

The vibrations are produced by for example the acceleration and deceleration of blood due to abrupt mechanical events of the cardiac cycle and their manifestation in the brain's neural and circulatory system.

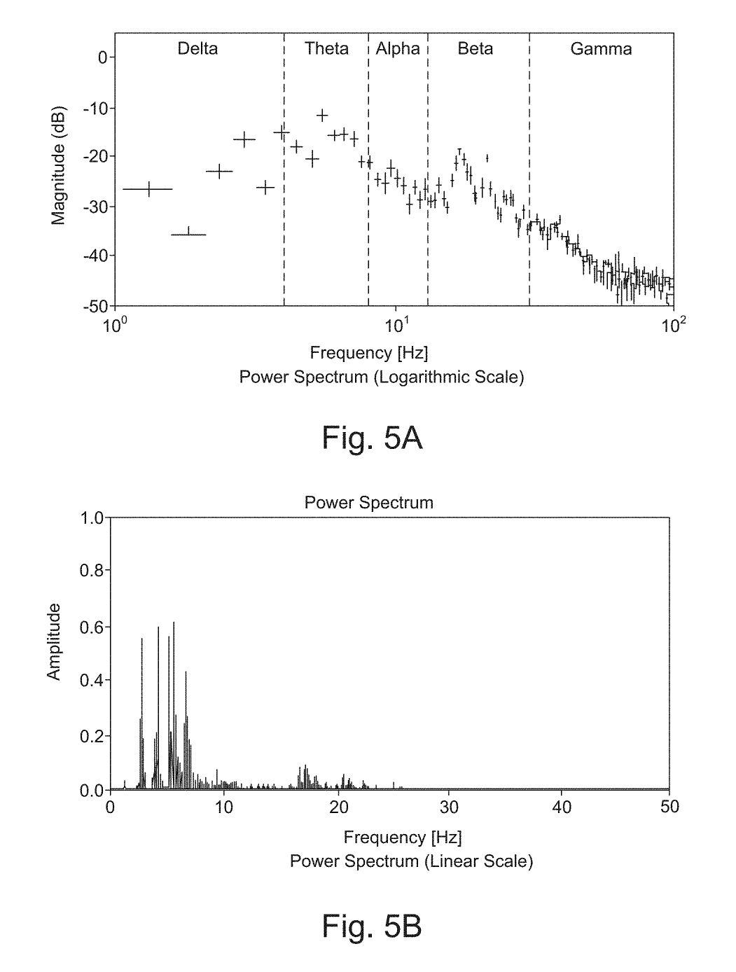

FIGS. 5A and 5B show the power spectrum of an acoustic signals measured inside a human ear canal of FIG. 4B. FIG. 5A has logarithmic scale. Dashed lines indicate ranges corresponding to different brain waves detectable with EEG. FIG. 5B shows the amplitude on a linear scale. Prominent peaks below 10 Hz correspond mostly to the cardiac cycle.

The high metabolic demand of neuronal tissue requires tight coordination between neuronal activity and blood flow within the brain parenchyma, known as functional hyperemia (see The Cerebral Circulation, by Marilyn J. Cipolla, Morgan & Claypool Life Sciences; 2009, https://wv.ncbi.nlm.nih.gov/books/NBK53081/). However, in order for flow to increase to areas within the brain that demand it, upstream vessels must dilate in order to avoid reductions in downstream microvascular pressure. Therefore, coordinated flow responses occur in the brain, likely due to conducted or flow-mediated vasodilation from distal to proximal arterial segments and to myogenic mechanisms that increase flow in response to decreased pressure.

Active brain regions require more oxygen, as such, more blood flows into more active parts of the brain. In addition, neural tissue can generate oscillatory activity —oscillations in the membrane potential or rhythmic patterns of action potentials. Sounds present at and in the ear canal are the result of blood flow, muscles and neural activity. As such, microphones placed in or near the ear canal can detect these acoustic signals. Detected acoustic signals can be used for example to infer the brain activity level, blood circulation, characterise cardiovascular system, heart rate, or even to determine the spatial origin of brain activity.

Human brain activity detected with EEG generally conforms to the 1/f ‘pink noise’ decay and is punctuated by prominent peaks in the canonical delta (0-4 Hz) and alpha (8-13 Hz) frequency bands (See, Spectral Signatures of Reorganised Brain Networks in Disorders of Consciousness, Chennu et al, Oct. 16, 2014, https://doi.org/10.1371/journal.pcbi.1003887)

In the typically case, the user 10 wears the head-mounted transducer system 100 such as earbuds or other earphones or another type of headset. The transducer system and its microphones or other acoustic sensors, i.e., sensors, measure acoustic signals propagating through the user's body. The acoustic sensors in the illustrated example are positioned outside or at the entrance to the ear canal, or inside the ear canal to detect body's infrasound and other acoustic signals.

The microphones best suited for this purpose are electret condensers as they have relatively flat responses in the infrasonic frequency range (See Response identification in the extremely low frequency region of an electret condenser microphone, Jeng, Yih-Nen et al., Sensors (Basel, Switzerland) vol. 11, 1 (2011): 623-37, https://www.ncbi.nlm.nih.gov/pubmed/22346594) and have low noise floors at low frequencies (A Portable Infrasonic Detection System, Shams, Qamar A. et al., Aug. 19, 2008, https://ntrs.nasa.gov/search.jsp?R=20080034649). A range of microphone sizes can be employed—from 2 millimeters (mm) up to 9 mm in diameter. A single large microphone will generally be less noisy at low frequencies, while multiple smaller microphones can be implemented to capture uncorrelated signals.

The detected sounds are outputted to the processing system 106 through for example Bluetooth, WiFi, or a wired connection 107. The controller module 104, possibly integrated into one or both of the earbuds (102,103) maintains the wireless data connection 107. At least some of the data analysis will often be performed using the processing system user device 106 or data can be transmitted to the web-based computer server system 109 functioning as a component of the processing system or processing can be shared between the user device 106 and the web-based computer server system 109. The detected output of the brain's sound may be processed at for example a computer, virtual server, supercomputer, laptop, etc., and monitored by software running on the computer. Thus, through this analysis, the user can have real-time insight into biometric activity and vital signs or can view the data later.

The plots of FIG. 3 show example data recorded using microphones placed in the ear canal. The data show the cardiac waveforms with prominent peaks corresponding to ventricular contractions 1303 with consistent detection in both right and left ear. The analysis of the cardiac waveform detected using a microphone placed in ear canal can be used to extract precise information related to cardiovascular system such as heart rate, heart rate variability, arrhythmias, blood pressure, etc.

The plots of FIGS. 5A and 5B shows an example of a power spectrum obtained from 30 seconds of data shown in FIG. 4B collected using microphones placed in the ear canal. The processing of the user's brain activity can result in estimation of the power of the signal for given frequency range. The detected infrasound can be processed by software, which determines further actions. For example, real-time data can be compared with previous user's data. The detected brain sound may also be monitored by machine learning algorithms by connecting to the computer, directly or remotely, e. g., through the Internet. A response may provide an alert on the user's smartphone or smartwatch.

The processing system user device 106 preferably has a user interface presented on a touch-screen display of the device, which does not require any information of a personal nature to be retained. Thus, the anonymity of the user can be preserved even when the body activity and vital signs are being detected. In such a case, the brain waves can be monitored by the earphones and the detected body sounds transmitted to the computer without any identification information being possessed by the computer.

Further, the user may have an application running on processing system user device 106 that receives the detected, and typically digitized, infrasound, processes the output of the head-mounted transducer system 100 and determines whether or not a response to the detected signal should be generated for the user.

The embodiments of the invention can also have additional microphones, the purpose of which is to detect external sources of the infrasound and audible sound. The microphones can be oriented facing away from one another with a variety of angles to capture sounds originating from different portions of a user's skull. The external microphones can be used to facilitate discrimination if identified acoustic signals originate from user activity or is a result of external noise.

Negative impacts from external infrasonic sources on human health have been extensively studied. Infrasounds are produced by natural sources as well as human activity. Example sources of infrasounds are planes, cars, natural disasters, nuclear explosions, air conditioning units, thunderstorms, avalanches, meteorite strikes, winds, machinery, dams, bridges, and animals (for example whales and elephants). The external microphones can also be used to monitor level and frequency of external infrasonic noise and help to determine its origin.

The biosensor system 50 can also include audio speakers that would allow for the generation of sounds like music in the audible frequency range. In addition, the headset can have embedded additional sensors, for example, a thermometer to monitor user's body temperature, a gyroscope and an accelerometer to characterize the user's motion.

While preferred embodiments have been set forth with specific details, further embodiments, modifications, and variations are contemplated according to the broader aspects of the present invention.

Earphones

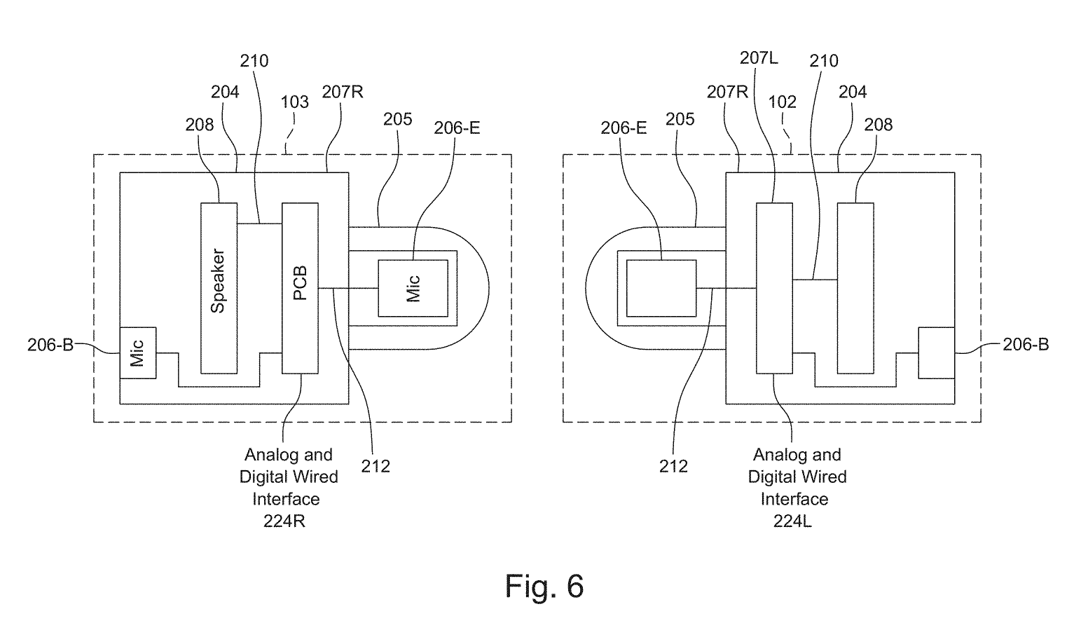

FIG. 6 shows one potential configuration for the left and right earbuds 102, 103.

In more detail, each of the earbuds 102, 103 includes an earbud housing 204. An ear canal extension 205 of the housing 204 projects into the ear canal of the user 10. The acoustic sensor 206-E for detecting acoustic signals from the user's body is housed in this extension 205.

In the illustrated example, a speaker 208 and another background acoustic sensor 206-B, for background and environment sounds, are provided near the distal side of the housing 204. Also within the housing is a printed circuit board (PCB) 207.

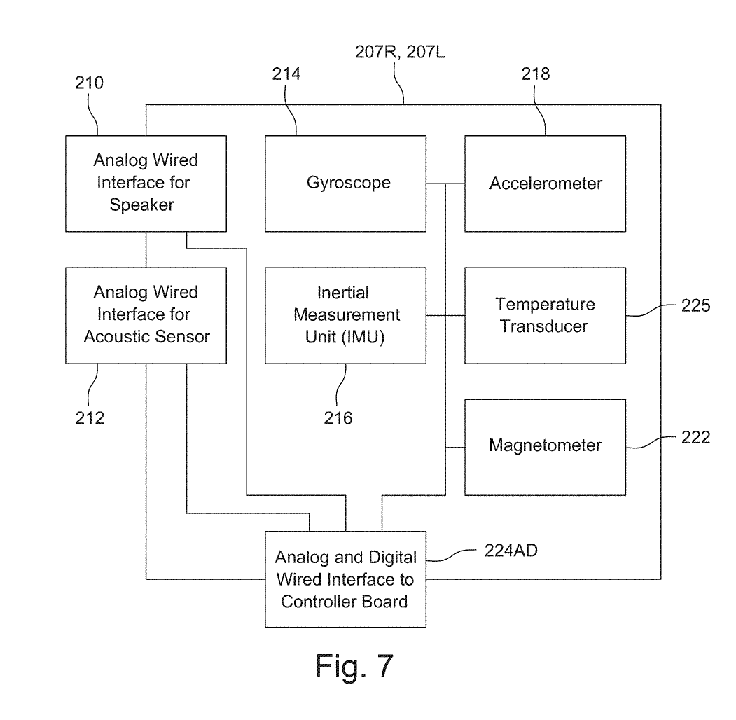

FIG. 7 is a block diagram showing potential components of the printed circuit board 207 for the each of the left and right earbuds 102, 103.

Preferably, each of the PCB 207L, 207R contains a gyroscope 214 for detecting angular rotation such as rotation of the head of the user 10. In one case, a MEMS (microelectromechanical system) gyroscope is installed on the PCB 207. In addition, a MEMS accelerometer 218 is included on the PCB 207 for detecting acceleration and also orientation within the Earth's gravitational field. A temperature transducer 225 is included for sensing temperature and is preferably located to detect the body temperature of the user 10. A magnetometer 222 can also be included for detecting the orientation of the earbud in the Earth's magnetic field.

Also, in some examples, an inertial measurement unit (IMU) 216 is further provided for detecting movement of the earbuds 102, 103.

The PCB 207 also supports an analog wired speaker interface 210 to the respective speaker 208 and an analog wired acoustic interface 212 for the respective acoustic sensors 206-E and 206-B. A combined analog and digital wired module interface 224AD connects the PCB 207 to the controller module 104.

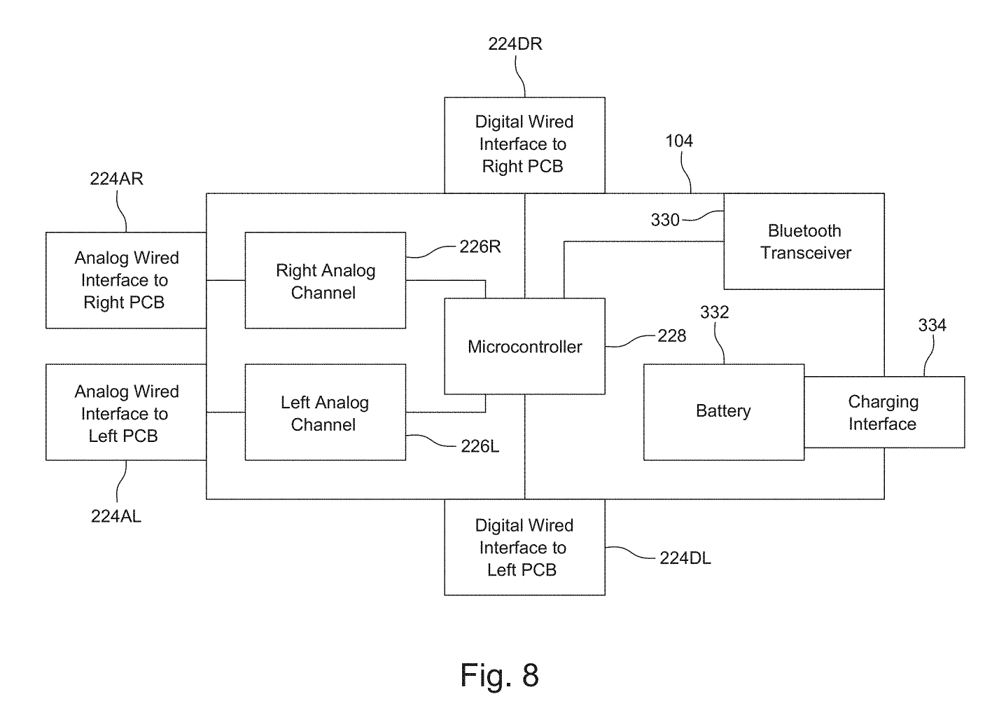

FIG. 8 is a block diagram showing the controller module 104 that connects to each of the left and right earbuds 102, 103.

In more detail, analog wired interface 224AR is provided to the PCB 207R for the right earbud 103. In addition, analog wired interface 224AL is provided to the PCB 207L for the left earbud 102. A right analog Channel 226R and a left analog Channel 226L function as the interface between the microcontroller 228 and the acoustic sensors 206-E and 206-B for each of the left and right earbuds 102, 103.

The right digital wired interface 224DR connects the microcontroller 228 to the right PCB 207R and a left digital wired interface 224DL connects the microcontroller 228 to the left PCB 207L. These interfaces allow the microcontroller 228 to power and to interrogate the auxiliary sensors including the gyroscope 214, accelerometer 218, IMU 216, temperature transducer 225, and magnetometer 222 of each of the left and right earbuds 102, 103.

Generally, the microprocessor 228 processes the information from both of the acoustic sensors and the auxiliary sensors from each of the earbuds 102, 103 and transmits the information to the processing system user device 106 via the wireless connection 107 maintained by a Bluetooth transceiver 330 that maintains the data connection.

In other embodiments, the functions of the processing system are built into the controller module 104.

Also provided in the controller module 104 is a battery 332 that provides power to the controller module 104 and each of the earbuds 102, 103 via the wired interfaces 224L, 224R.

In addition, information is received from the processing system user device 106 via the Bluetooth transceiver 330 and then processed by the microcontroller 228. For example, audio information to be reproduced by the respective speakers 208 for each of the respective earbuds 102 and 103 is typically transmitted from the processing system user device 106 and received by the Bluetooth transceiver 330. Then the microcontroller 228 provides the corresponding audio data to the right analog channel 226R and the left analog channel 226L.

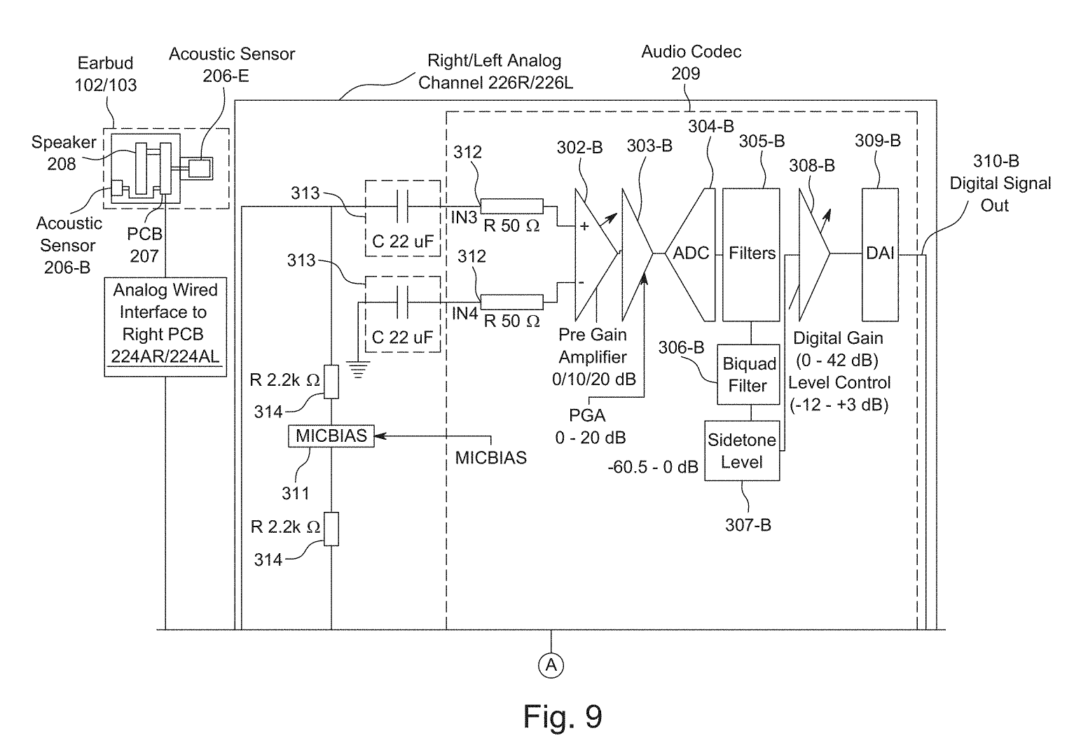

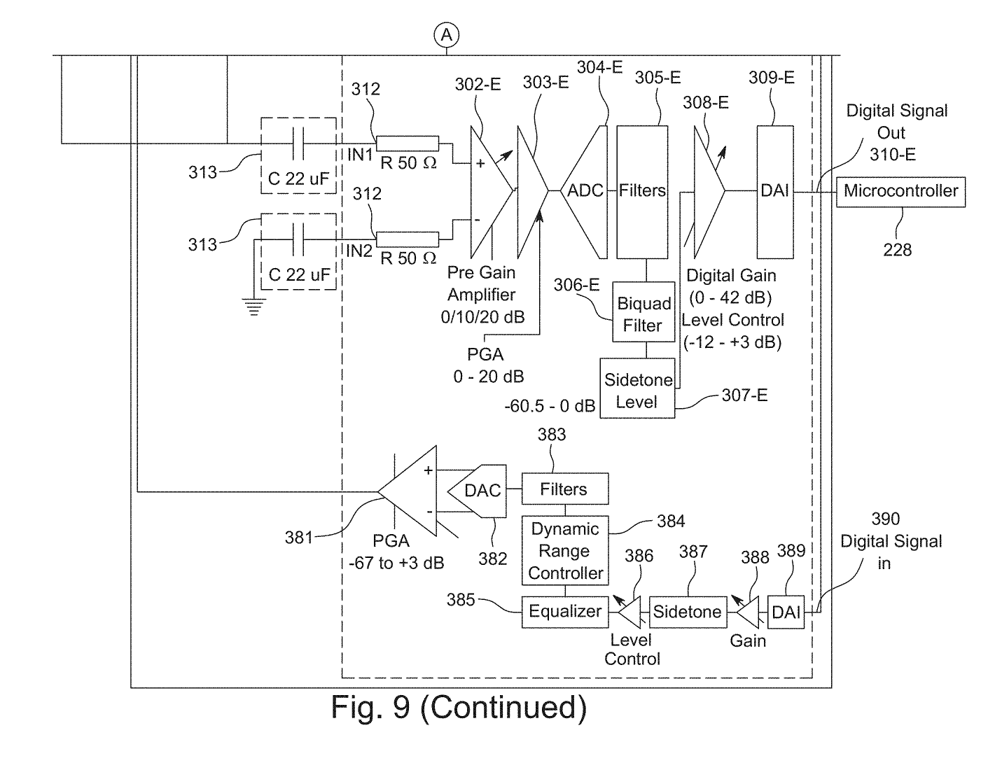

FIG. 9 is a circuit diagram showing an example circuit for each of the right analog channel 226R and the left analog channel 226L.

In more detail, each of the right and left analog channels 226R, 226L generally comprise a sampling circuit for the analog signals from the acoustic sensors 206-E and 206-B of the respective earbud and an analog drive circuit for the respective speaker 208.

In more detail, the analog signal from the acoustic sensors 206-E and 206-B are biased by a micbias circuit 311 through resistors 314. DC blocking capacitors 313 are included at the inputs of Audio Codec 209 for the acoustic sensors 206-B and 206-E. This DC filtered signal from the acoustic sensors is then provided to the Pre Gain Amplifier 302-E/302-B.

The Pre Gain Amplifier 302-E/302-B amplifies the signal to improve noise tolerance during processing. The output of 302-E/302-B is then fed to a programmable gain amplifier (PGA) 303-E/303-B respectively. This amplifier (typically an operational amplifier) increases the signal amplitude by applying a variable gain, or amplification factor. This gain value can be varied by software using the microcontroller 228.

The amplified analog signal from the PGA 303-E/303-B is then digitized by the Analog-to-Digital convertor (ADC) 304-E/304-B. To modify this digital signal as per need, two filters are applied, Digital filter 305-E/305-B and Biquad Filter 306-E/306-B. A Sidetone Level 307-E/307-B is also provided to allow the signal to be directly sent to the connected speaker, if required. This digital signal is then digitally amplified by the Digital Gain and Level Control 308-E/308-B. The output of 308-E/308-B is then converted to appropriate serial data format by the Digital Audio Interface (DAI) 309-E/309-B and this serial digital data 310-E/310-B is sent to the microcontroller 228.

On the other hand, digital audio 390 from the microcontroller 228 is received by DAI 389. The output has its level controlled by a digital amplifier 388 under control of the microcontroller 228. A sidetone 387 along with a level control 386 are further provided. An equalizer 385 changes the spectral content of the digital audio signal under the control of the microcontroller 228. Further, a dynamic range controller 384 controls dynamic range. Finally, digital filters 383 are provided before the digital audio signal is provided to a digital to analog converter 382. A drive amplifier 381 powers the speakers 208 in response to the analog signal from the DAC 382.

The capacitor 313 should have sufficiently high capacitance to allow infrasonic frequencies to reach the first amplifier while smoothing out lower frequency large time domain oscillations in the microphone's signal. In this way, it functions as a high pass filter. The cut off at low frequencies is controlled by capacitor 313 and the resistor 312 such that signals with frequencies f<1/(2πR C) will be attenuated. Thus, capacitor 313 and resistor 312 are chosen such that the cut off frequency (f) is less than 5 Hz and typically less than 2 Hz and preferably less than 1 Hz. Therefore frequencies higher than 5 Hz, 2 Hz, and 1 Hz, respectively pass to the respective amplifiers 302-B, 302-E. In fact, in the present embodiment of the invention, the values of the capacitor 313 and resistor 312 are the capacitor C=22 uF and resistor R=50 k Ohm, which give the cut off frequency of ˜0.1 Hz. Therefore, presently, the cut off frequency is less than 1 Hz and generally it should be less than 0.1 Hz, f<<1 Hz. The two remaining resistors 314 are connected to MICBIAS 311 and ground, respectively, have values that are chosen to center the single at ½ of the maximum of the voltage supply.

The acoustic sensors 206-E/206-B may be one or more different shapes including, but not limited to, circular, elliptical, regular N-sided polygon, an irregular N-sided polygon. A variety of microphone sizes may be used. Sizes of 2 mm-9 mm can be fitted in the ear canal. This variety of sizes can accommodate users with both large and small ear canals.

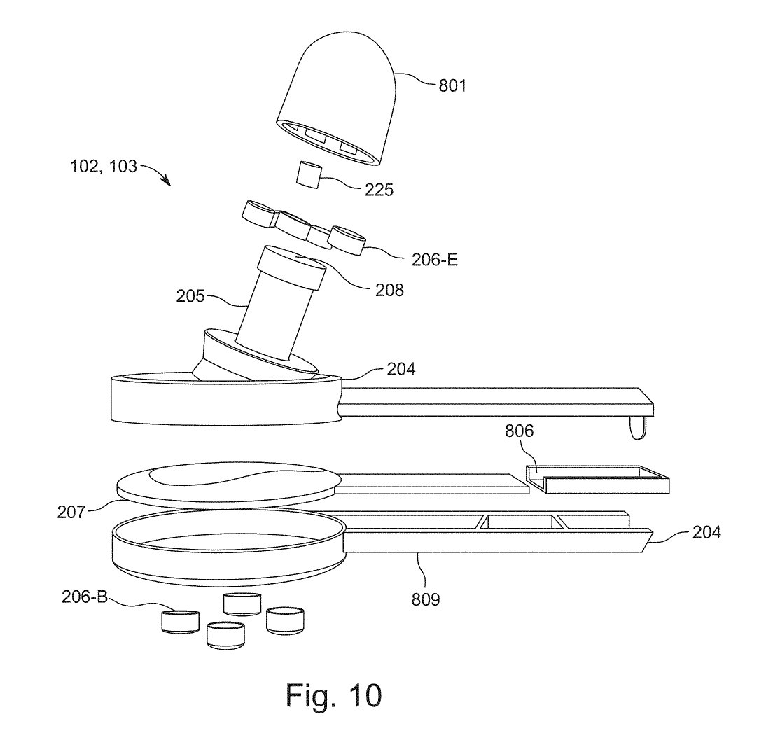

Referring to FIG. 10 there is depicted an exemplary earbud 102, 103 of the head-mounted transducer system 100 in accordance with some embodiments of the invention such as depicted in FIG. 1.

An earbud cover 801 is placed over the ear canal extension 205. The cover 801 can have a different shapes and colors and can be made of different materials such as rubber, plastics, wood, metal, carbon fiber, fiberglass, etc.

The earbud 102, 103 has an embedded temperature transducer 225 which can be an infrared detector. A typical digital thermometer can work from −40 C to 100 C with an accuracy of 0.25 C.

In the exemplary system, SDA and SCL pins use the I2C protocol for communication. Such a configuration allows multiple sensors to attach to the same bus on the microcontroller 228. Once digital information is passed to the microcontroller 228 with the SDA and SCL pins, the microcontroller translates the signals to a physical temperature using an installed reference library, using reference curves from the manufacturer of the thermometer. The infrared digital temperature transducer 225 can be placed near the ear opening, or within the ear canal itself. It is placed such that it has a wide field of view to areas of the ear which give accurate temperature reading such as the interior ear canal. The temperature transducer 225 may have a cover to inhibit contact with the user's skin to increase the accuracy of the measurement.

A microphone or an array of acoustic sensors 206-E/206-B are used to enable the head-mounted transducer system 100 to detect internal body sounds and background sound. The microphone or microphones 206-E for detecting the sounds from the body can be located inside or at the entrance to the ear canal and can have different locations and orientations. The exemplary earphone has a speaker 208 that can play sound in audible frequency range and can be used to playback sound from another electronic device.

The earphone housing 204 is in two parts having a basic clamshell design. It holds different parts and can have different colors, shapes, and can be produced of different materials such as plastics, wood, metal, carbon fiber, fiberglass, etc.

Inside the earphone housing 204 there is possibly a battery 806 and the PCB 207. The battery 806 can be for example a lithium ion. The PCB 207 comprises circuits, for example, as the one shown in FIG. 7. In addition, in some embodiments the control module 226 is further implemented on the PCB 207.

The background external microphone or array of microphones 206-B is preferably added to detect environmental sounds in the low frequency range. The detected sounds are then digitized and provided to the microcontroller 228.

The combination of microphone placement and earbud cover 801 can be designed to maximize the Occlusion Effect (The “Occlusion Effect”—What it is and What to Do About it, Mark Ross, January/February 2004, https://web.archive.org/web/20070806184522/http:/www.hearingresearch.org/Dr. Ross/occlusion.htm) within the ear canal which provides up to 40 dB of amplification of low frequency sounds within the ear canal. The ear can be partially or completely sealed with the earbud cover 801, and the placement of the 801 within the ear canal can be used to maximize the Occlusion Effect with a medium insertion distance (Bone Conduction and the Middle Ear, Stenfelt, Stefan. (2013).10.1007/978-1-4614-6591-1_6., https://www.researchgate.net/publication/278703232_Bone_Conduction_and_the_Middle_Ear).

The accelerometer 218 on the circuit board 207 allows for better distinction of the origin of internal sound related to the user's motion. An exemplary accelerometer with three axis (x,y,z) attached to the PCB 207 or it could be embedded into the microcontroller 228. The exemplary accelerometer 218 can be analog with three axis (x,y,z) attached to the microcontroller 228. The accelerometer 218 can be placed in the long stem-like section 809 of the earbud 102, 103. The exemplary accelerometer works by a change in capacitance as acceleration moves the sensing elements. The output of each axis of the accelerometer is linked to an analog pin in the microcontroller 228. The microcontroller can then send this data to the user's mobile device or the cloud using WiFi, cellular service, or Bluetooth. The microcontroller 228 can also use the accelerometer data to perform local data analysis or change the gain in the digital potentiometer in the right analog channel 226R and the left analog channel 226L shown in FIG. 9.

The gyroscope 214 on the PCB 207 is employed as an auxiliary motion detection and characterization system. Such gyroscope can be a low power with three axis (x,y,z) attached to the microcontroller 228 will be embedded into PCB 207. The data from the gyroscope 214 can be sent to the microcontroller 228 using for example the 12C protocol for digital gyroscope signals. The microcontroller 228 can then send the data from each axis of the gyroscope to the user's mobile device processing system 106 or the cloud computer server system 109 using WiFi, cellular service, or Bluetooth. The microcontroller 228 can also use the gyroscope data to perform local data analysis or change the gain in thein the right analog channel 226R and the left analog channel 226L shown in FIG. 9.

Data Acquisition System

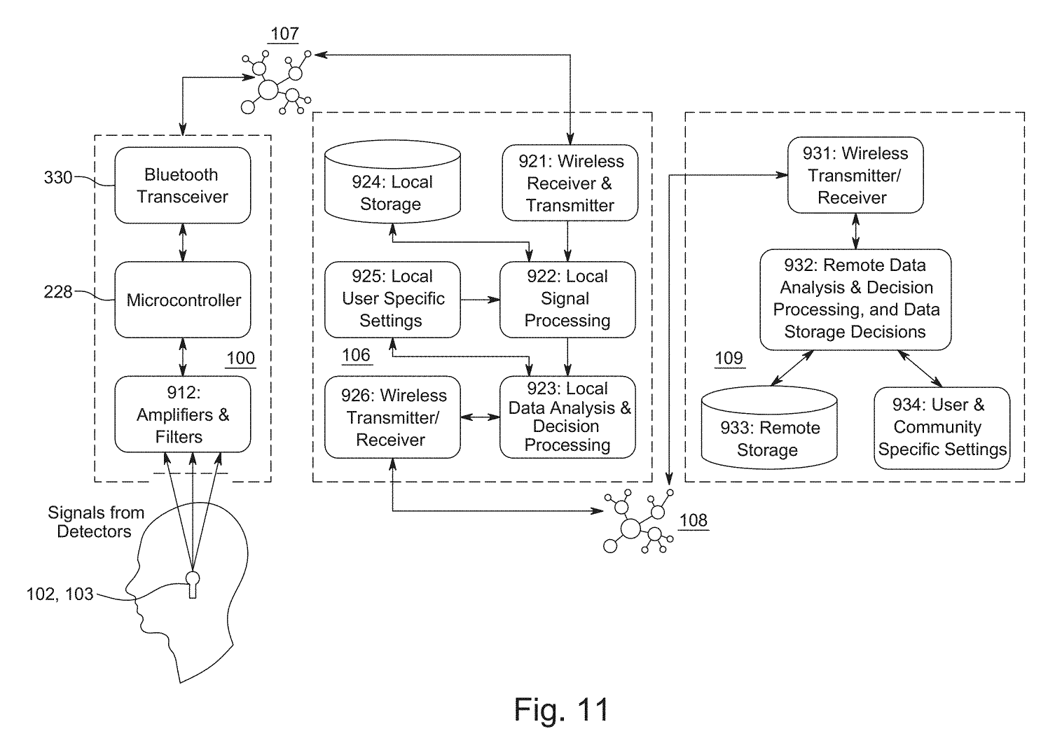

FIG. 11 depicts a block diagram illustrating the operation of the biosensor system 50 according to an embodiment of the invention. The biosensor system 50 presented here is an exemplary way for processing biofeedback data from multiple sensors embedded into a headset or an earphone system of the head-mounted transducer system 100.

The microcontroller 228 collects the signals from sensor array 911 including, but not limited to acoustic transducers, e.g., microphones 206-E/206-B, gyroscope 214, accelerometer 218, temperature transducer 225, magnetometer 222, and/or the inertial measurement unit (IMU) 216.

The data can be transmitted from sensor array 911 to filters and amplifiers 912. The filters 912 can for example be used to filter out low or high frequency to adjust signal to desired frequency range. The amplifiers 912 can have an adjustable gain for example to avoid signal saturation caused by an intense user motion. The gain level could be estimated by the user device 106 and transmitted back to the microcontroller 228 through the wireless receivers and transmitters. The amplifiers and filters 912 connect to a microcontroller 228 which selects which sensors are to be used at any given time. The microcontroller 228 can sample information from sensors 911 at different time intervals. For example, temperature can be sampled at lower rate as compared to acoustic sensors 206-E and 206-B. The microcontroller 228 sends out collected data via the Bluetooth transceiver 330 to the processing system user device 106 and takes inputs from processing system user device 106 via the Bluetooth transceiver 330 to adjust the gain in the amplifiers 912 and/or modify the sampling rate from data taken from the sensor array 911. Data is sent/received in the microcontroller with the Bluetooth transceiver 330 via the link 107.

The data are sent out by the microcontroller 228 of the head mounted transducer system 100 via the Bluetooth transceiver 330 to the processing system user device 106. A Bluetooth transceiver 921 supports the other end of the data wireless link 107 for the user device 106.

A local signal processing module 922 executes on the central processing unit of the user device 106 and uses data from the head-mounted transducer system 110 and may combine it with data stored locally in a local database 924 before sending it to the local analysis module 923, which typically also executes on the central processing unit of the user device 106.

The local signal processing module 922 usually decides what fraction of data is sent out to a remote storage 933 of the cloud computer server system 109. For example, to facilitate the signal processing, only number of samples N equal to the next power of two could be sent. As such, from 1−(N−1) samples data are sent from the local signal processing unit 922 to the local storage 924, and on the Nth sample data are sent from the local storage 924 back to the local signal processing unit 922 to combine the 1−(N−1) data samples with the Nth data sample to send them all along to the local analysis module 923. The way in which data are stored/combined can depend on local user settings 925 and the analysis coupling 923. For example, the user can turn off thermometer. The option to turn off given sensor can be specified in the local user specific settings 925. As a result of switching off one of the sensors, the data could be stored less frequently if it would not impede with the calculations needed by the local data analysis unit 923.

The local data analysis and decision processing unit 923 decides what data to transmit to the cloud computer server system 109 via a wide area network wireless transmitter 926 that supports the wireless data link 108 and what data to display to the user. The decision on data transmission and display is made based on information available in the local user settings in 925, or information received through the wireless transmitter/receiver 926, from the cloud computer server system 109. For example, data sampling can be increased by the cloud computer server system 109 in a geographical region where an earthquake has been detected. In such a case, the cloud computer server system 109 would send a signal from the wireless transmitter 931 to the user device 106 via its transceiver 926, which would then communicate with local data analysis and decision process module 923 to increase sampling/storage of data for a specified period of time for users in that region. This information could then also be propagated to the head-mounted transducer system to change the sampling/data transfer rate there. In principle, other data from the user device 106 like the user's geographical location, information about music that users are listening to, other sources could be combine at the user device 106 or the cloud computer server system 109 levels.

The local storage 924 can be used to store a fraction of data for a given amount of time before either processing it or sending it to the server system 109 via the wireless transmitter/receiver 926.

In accordance with some embodiments of the invention, the wireless receiver and transmitter 921 may include, but is not limited to Bluetooth transmitter/receiver that can handle communication with the transducer system 100. While the wireless transmitter/receiver 926 can be based on a communication using WiFi that would for example transmit data from/to the user device 106 and/or the cloud server system 109, such as, for example the cloud based storage.

The wireless transmitter/receiver 926 will transmit processed data to the cloud server system 109. The data can be transmitted using Bluetooth or a WiFi or a wide area network (cellular) connection. The wireless transmitter/receiver 926 can also take instructions from the cloud server system 109. Transmission will happen over the network 108.

The cloud server system 109 also stores and analyze data, functioning as an additional processing system, using, for example, servers, supercomputers, or in the cloud. The wireless transceiver 931 gets data from the user device 106 shown and hundreds or thousands of other devices 106 of various subscribing users and transmits it to a remote signal processing unit 932 that executes on the servers.

The remote signal processing unit 932, typically executing on one or more servers, can process a single user's data and combine personal data from the user and/or data or metadata from other users to perform more computationally intensive analysis algorithms. The cloud server system 109 can also combine data about a user that is stored in a remote database 934. The cloud server system 109 can decide to store all or some of the user's data, or store metadata from the user's data, or combine data/metadata from multiple users in a remote storage unit 933. The cloud server system 109 also decides to send information back to the various user devices 106, through the wireless transmitter/receiver 931. The cloud server system 109 also deletes data from the remote storage 933 based on user's preferences, or a data curation algorithm. The remote storage 933 can be a long-term storage for the whole system. The remote storage 933 can use cloud technology, servers, or supercomputers. The data storage on the remote storage 933 can include raw data from users obtained from the head mounted transducer systems 100 over the various users, preprocessed data the respective user devices 106 and data specified according to user's preferences. The user data can be encrypted and can be backed up.

It is an option of the system that users can have a multiple transducer systems 100 that would connect to the same user device 106 or multiple user devices 106 that would be connected to user account on the data storage facility 930. The user can have a multiple sets of headphones/earbuds equipped with biosensors that would collect data into one account. For example, a user can have different designs of bio-earphones depending on their purpose, for example earphones for sleeping, meditating, sport, etc. A user with multiple bio-earphones would be allowed to connect to multiple bio-earphones using the same application and account. In addition, a user can use multiple devices to connect to the same bio-earphones or the same accounts.

The transducer system 100 has its own storage capability in some examples to address the case where it becomes disconnected from its user device 106. In case of lack of connection between the transducer system 100 and the user device 106, the data is preferably buffered and stored locally until the connection is re-established. If the local storage runs out of space, the older or newer data would be deleted according with users' preferences. The microcontroller 228 could have a potential to process the un-transmitted data into more compact form and send to the user device 106 once the connection is re-established.

Data Analysis

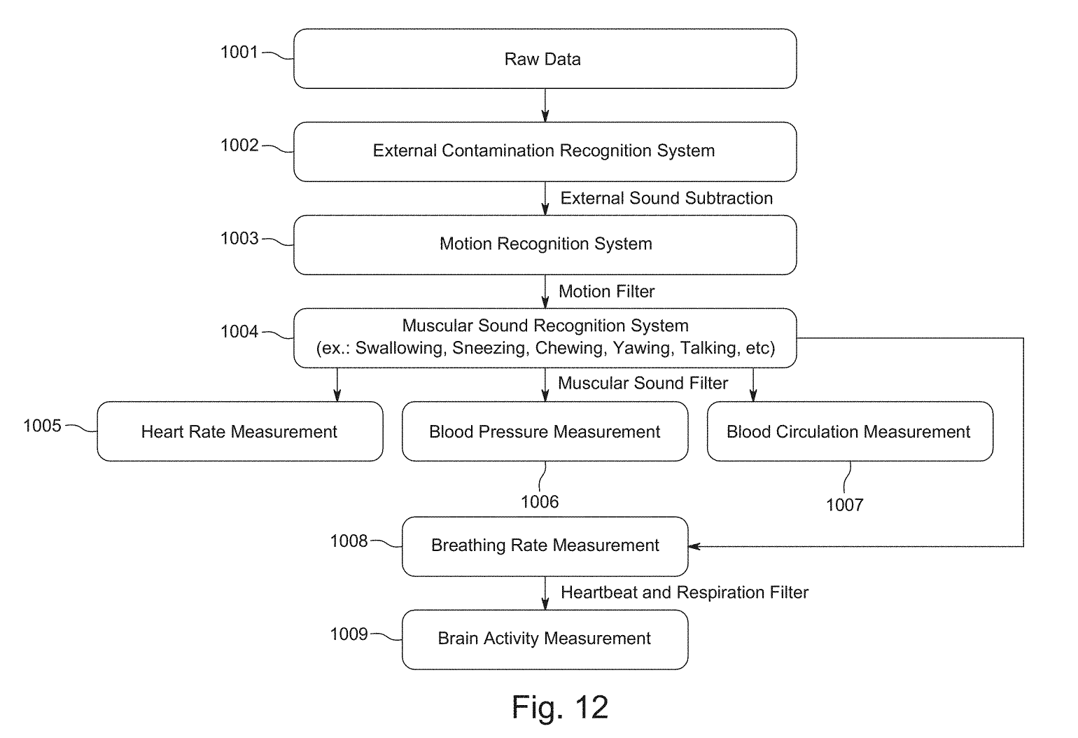

FIG. 12 depicts an exemplary flowchart for signal processing of biosensor data according to an embodiment of the invention.

Raw data 1001 are received from sensors 911 including but not limited to acoustic transducers, e.g., microphones 206-E/206-B, gyroscope 214, accelerometer 218, temperature transducer 225, magnetometer 222, and/or the inertial measurement unit (IMU) 216. The data are analyzed in multiple steps.

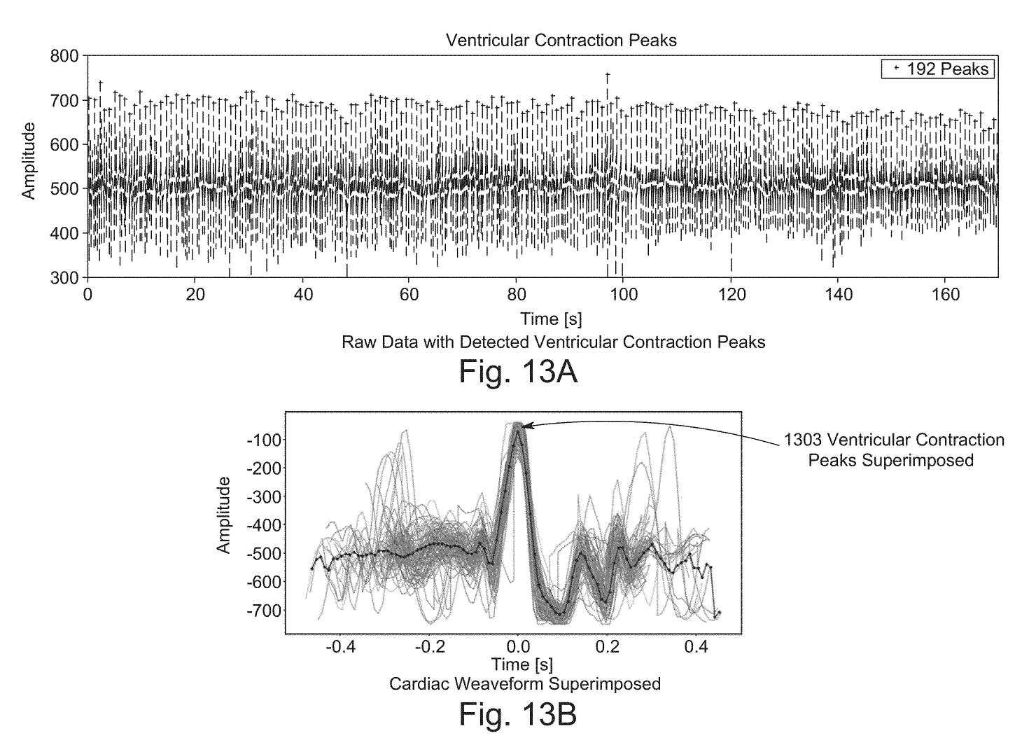

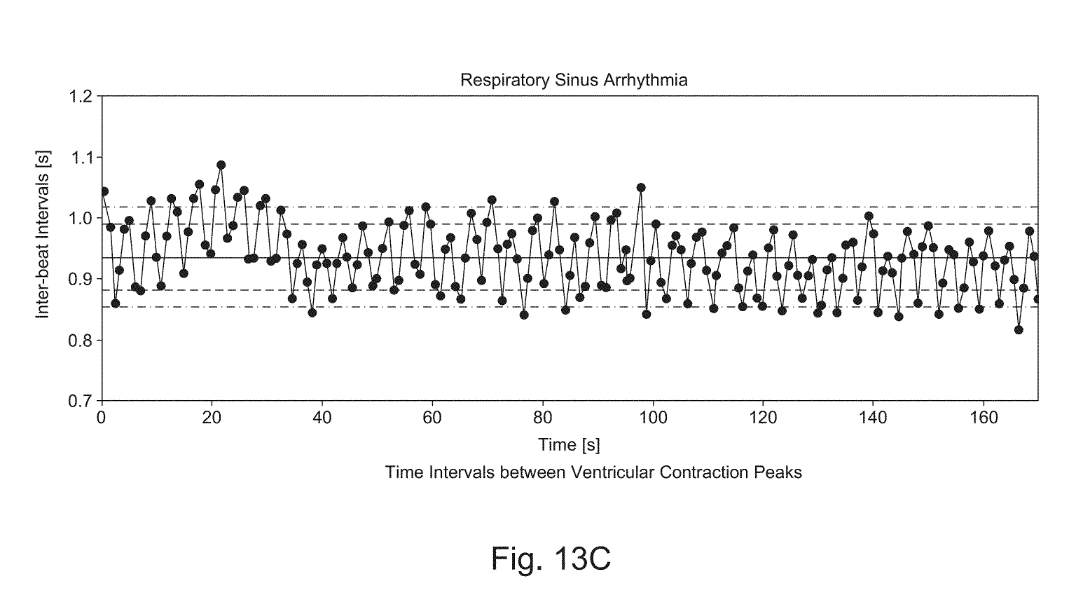

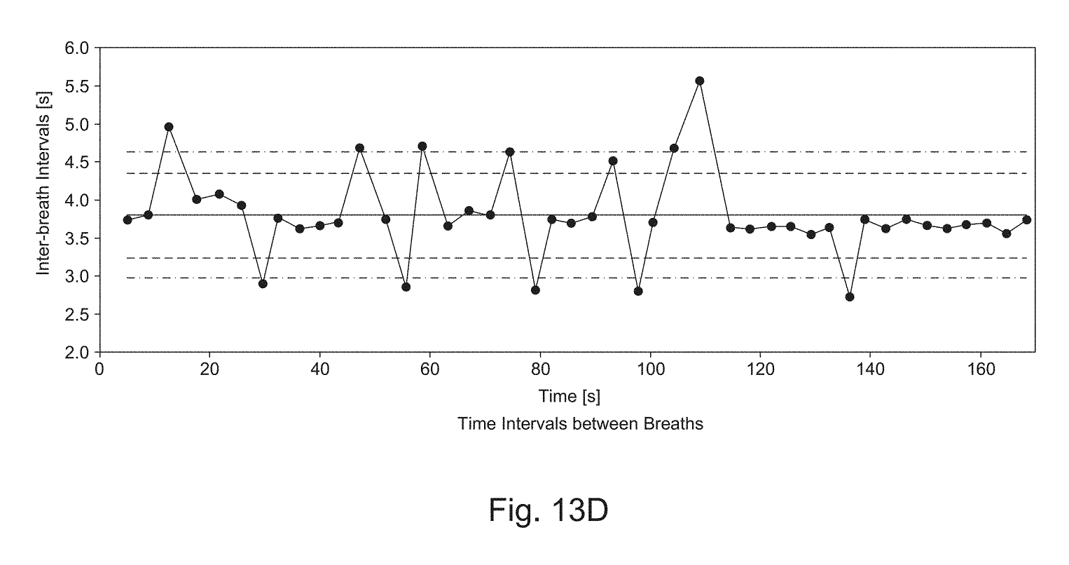

The data sampling is chosen is such a way to reconstruct the cardiac waveform as shown in FIG. 13B. In the embodiment of the invention, the sampling rate range was between 100 Hz and 1 kHz. Preferably, the sampling rate is around 100 Hz and generally should not be less than 100 Hz. Moreover, to collect high fidelity data to better model the cardiac waveform and extract detail biofeedback information, like for example blood pressure, the sampling rate should be greater than 100 Hz.

In the embodiment of the invention, the circuit as presented in FIG. 9 allows infrasonic frequencies greater than 0.1 Hz to pass, which enables signal of cardiac activity to be detected. In addition, when a user 10 is using audio speakers 208, the audio codec 209 can be configured to filter out a potential signal interference generated by the speaker 208 from the acoustic sensors 206-E and 206-B.

After amplification and initial filtering 912 of FIG. 11, data are processed and stored in other units including but not limited to the microcontroller 228, the local signal processing module 922, the local data analysis and decision processing module 923, and remote data analysis and decision processing module 932. The data are typically sent every few seconds into series of, for example, overlapping 10-second long data sequences. The length of, overlapping window, and the number of samples within each sequence may vary in other embodiments.

When an array of microphones is used, the voltage of the microphones can be added before analysis. The signal from internal and external arrays of microphones is analyzed separately. Signal summation immediately improves the signal to noise ratio. The microphone data are then calibrated to achieve a signal in physical units (dB). Each data sample from the microphones is pre-processed in preparation for Fast Fourier Transform (FFT). For example, the mean is subtracted from the data, a window function is applied, etc. Also, Wavelet Filters can be used.

An external contamination recognition system 1002 uses data from microphones located inside or at the entrance to the ear canal 206-E and external acoustic sensor 206-B. The purpose of external acoustic sensor 206-B is to monitor and recognize acoustic signals including infrasounds originating from the user's environment and distinguishing them from acoustic signals produced by human body. Users can access and view the spectral characteristics of external environmental infrasound. Users can choose in the local user specific setting 925 to be alerted about an increased level of infrasound in the environment. The local data analysis system 923 can be used to provide basic identification of a possible origin of the detected infrasound. The data from external microphones can also be analyzed in more depth by the remote data analysis system 932, where data can be combined with information collected from other users. The environmental infrasound data analyzed from multiple users in common geographical area can be used to detect and warn users about possible dangers, such as earthquakes, avalanches, nuclear weapon tests, etc.

Frequencies detected by the external/background acoustic sensor 206-B are filtered out from the signal from internal acoustic sensor 206-E. Body infrasound data with subtracted external infrasounds are then processed by the motion recognition system 1003, where the motion detection is supported by an auxiliary set of sensors 911 including by not limited to an accelerometer 218 and gyroscope 214. The motion recognition system 1003 provides a means of detecting if the user is moving. If no motion is detected the data sample is marked as “no motion.” If motion is detected, then the system performs further analysis to characterize the signal. The data are analyzed to search for patterns that correspond to different body motions including but not limited to walking, running, jumping, getting up, sitting down, falling, turning, head movement, etc.

Data from internal 206-E and external 206-B acoustic sensors can be combined with data from accelerometers 218 and gyroscopes 214. If adjustable gain is used, then the current level of the gain is another data source that can be used. Data from microphones can also be analyzed separately. The motion can be detected and characterized using, for example, wavelet analysis, the Hilbert-Huang transform, empirical mode decomposition, canonical correlation analysis, independent component analysis, machine learning algorithms, or some combination of methodologies. The infrasound corresponding to motion is filtered out from data, or data corresponding to period of an extensive motion are excluded from the analysis.

Data sample with the filtered user's motion or data samples marked as “no motion” are further analyzed by the muscular sound recognition system 1004. The goal of the system 1004 is to identify and characterize stationary muscle sounds such as swallowing, sneezing, chewing, yawning, talking, etc. The removal of artifacts, e.g., muscle movement, can be accomplished via similar methodologies to those used to filter out user motion. Artifacts can be removed using, for example, wavelet analysis, empirical mode decomposition, canonical correlation analysis, independent component analysis, machine learning algorithms, or some combination of methodologies. Data samples with too high muscle signal that cannot be filtered out are excluded from analysis. The data with successfully filtered out muscle signals or identified as containing no muscle as no muscle signal contamination are marked as “muscle clean” and are used for further analysis.

The “muscle clean” data are run through a variant of the Discrete Fourier Transform, e.g. a Fast Fourier Transform (FFT) within some embodiment of the invention, to decompose the origin of the signal into constituent heart rate 1005, blood pressure 1006, blood circulation 1007, breathing rate 1008, etc.

With reference back, FIG. 3 shows 10 seconds of acoustic body activity recorded with a microphone located inside the ear canal. This signal demonstrates that motion and muscle movement can be detected and is indicated as loud signal 1302. The peaks with large amplitudes correspond to the ventricular contractions 1303. The heart rate 1005 can be extracted by calculating intervals between peaks corresponding to the ventricular contractions, which can be find by direct peak finding methods for data like shown in 1301. Heart rate can be also extracted by using FFT based methods or template methods by cross-correlating averaged cardiac waveform 302.

FIG. 4 show a one second of infrasound recorded with a microphone located inside the ear canal. The largest peak around 0.5 second correspond to the cardiac cycle maximum. Cerebral blood flow is determined by a number of factors, such as viscosity of blood, how dilated blood vessels are, and the net pressure of the flow of blood into the brain, known as cerebral perfusion pressure, which is determined by the body's blood pressure. Cerebral blood vessels are able to change the flow of blood through them by altering their diameters in a process called autoregulation—they constrict when systemic blood pressure is raised and dilate when it is lowered (https://en.wikipedia.org/wiki/Cerebral_circulation#cite_note-Kandel-6). Arterioles also constrict and dilate in response to different chemical concentrations. For example, they dilate in response to higher levels of carbon dioxide in the blood and constrict to lower levels of carbon dioxide. The amplitude, the rise and decay of heart beat depends on the blood pressure. Thus, the shape of the cardiac waveform 1301 detected by the processing system 106 using infrasound which can be used to extract the blood pressure in step 1006. To obtain better accuracy, the estimated blood pressure may be calibrated using an external blood pressure monitors.

Cerebral circulation is a blood circulation which arises in system of vessels of a head and spinal cord. Without significant variation between wakefulness or sleep or levels of physical/mental activity, the central nervous system uses some 15-20% of one's oxygen intake and only a slightly lesser percentage of the heart's output. Virtually all of this oxygen use is for conversion of glucose to CO2. Since neural tissue has no mechanism for the storage of oxygen, there is an oxygen metabolic reserve of only about 8-10 seconds. The brain automatically regulates the blood pressure between a range of about 50 to 140 mm Hg. If pressure falls below 50 mm Hg, adjustments to the vessel system cannot compensate, brain perfusion pressure also falls, and the result may be hypoxia and circulatory blockage. Pressure elevated above 140 mm Hg results in increased resistance to flow in the cerebral arterial tree. Excessive pressure can overwhelm flow resistance, leading to elevated capillary pressure, loss of fluid to the meager tissue compartment, and brain swelling. Blood circulation produces distinct sound frequencies depending on the flow efficiency and its synchronization with the heart rate. The blood circulation in step 1007 is measured as synchronization factor.

The heartbeat naturally varies with the breathing cycle, this phenomena is seen in a respiratory sinus arrhythmia (RSA). The relationship between the heartbeat rate and the breathing cycle is such that heartbeat amplitude tends to increase with inhalation and decrease with exhalation. As such, the amplitude and frequency of the heart rate variability pattern relates strongly to the depth and frequency of breathing (https://coherence.com/science_full_html j,roduction.htm). Thus, the RSA (see 13C) is used as an independent way of measuring breathing rate in step 1008, as further demonstrated in following sections (see FIG. 13D).

Heart and Breathing Rates: Algorithm

The following discussion describe the process performed by the processing system including usually the user device 106 and/or the server system 109, for example, to resolve the cardiac waveform and the respiratory rate based on the sensor data from the sensor 911 of the head-mounted transducer system 100 and possibly additional transducers located elsewhere on the user's body.

In more detail, each heart cycle comprises of atrial and ventricular contraction, as well as, blood ejection into the great vessels (see FIGS. 3,4, and 13). Other sounds and murmurs can indicate abnormalities. The distance between two sounds of ventricular contraction is the duration of one heart cycle is used to determine the heart rate by the processing system 106/109. One way to detect peaks (local maxima) or valleys (local minima) in data is for the processing system 106/109 to use the property that a peak (or valley) must be greater (or smaller) than its immediate neighbors. The ventricular contraction peaks shown in FIG. 13A can be detected by the processing system 106/109 by searching a signal in time for peaks requiring a minimum peak distance (MPD), peak width and a normalized threshold (only the peaks with amplitude higher than the threshold will be detected). The MPD parameter can vary depending on the user's heart rate. The algorithms may also include a cut on the width of the ventricular contraction peak estimated using the previously collected user's data or superimposed cardiac waveforms shown in FIG. 13B.

The peaks of FIG. 13A were detected by the processing system 106/109 using the minimum peak distance of 0.7 seconds and the normalized threshold of 0.8. The resolution of the detected peaks can be enhanced by the processing system 106/109 using interpolation and fitting a Gaussian near each previously detected peak. The enhanced positions of the ventricular contraction peaks are then used by the processing system 106/109 to calculate distances between the consecutive peaks. Such calculated distances between the peaks are then used by the processing system 106/109 to estimate the inter-beat intervals shown in FIG. 13C, which are used to obtain the heart rate. The positions of the peaks can also be extracted using a method incorporating, for example, continuous wavelet transform-based pattern matching. In the example shown in FIG. 13A, the processing system 106/109 determines that the average heart rate is 63.73+/−7.57 BPM, where the standard deviation reflects the respiratory sinus arrhythmia effect. The inter-beat intervals as a function of time shown in FIG. 13C are used by the processing system 106/109 to detect and characterize heart rhythms such as the respiratory sinus arrhythmia. The standard deviation is used by the processing system 106/109 to characterize the user's physical and emotional states, as well as, quantify heart rate variability. The solid line shows the average inter-beat interval in seconds. The dashed and dashed-dotted lines show inter-beat interval at 1 and 1.5 standard deviations, respectively. The estimated standard deviation can be used to detect and remove noise in the data as the one seen in FIG. 13A around 95 seconds.

The inter-beat interval shown in FIG. 13C shows a very clear respiratory sinus arrhythmia. The heart rate variability pattern relates strongly to the depth and frequency of breathing. Thus, to measure breathing rate, the processing system 106/109 uses the algorithm to detect peaks in the previously estimated heart rates. In the example presented in FIG. 13A, the heart rate amplitude were searched by the processing system 106/109 for within a minimum distance of two heartbeats and with a normalize amplitude above a threshold of 0.5. The distances between peaks in heart rate correspond to breathing. This estimated breathing duration is used to estimate the breathing rate of FIG. 13D.

In the presented example, the average respiration rate is 16.01+−2.14 breaths per minute. The standard deviation, similar to the case of the heart rate estimation, reflects variation in the user's breathing and can be used by the processing system 106/109 to characterize the user's physical and emotional states.

FIGS. 5A and 5B shows a power spectrum of an example infrasound signal measured inside a human ear canal, where prominent peaks below 10 Hz correspond mostly to the cardiac cycle.

Breathing induces vibrations which are detected by microphones 206-E located inside or at the entrance to ear canal. The breathing cycle is detected the processing system 106/109 by running FFT on a few second long time sample with a moving window at a step much smaller than the breathing time. This step allows the processing system 106/109 to monitor frequency content variable with breathing. The increased power in the frequency range above 20 Hz corresponds to an inhale, while decrease power indicates an exhale. The breathing rate and its characteristics are estimated by the processing system 106/109 by cross-correlating breathing templates with the time series. The breathing signal is further removed from the time series. The extracted heart beat peaks shown in FIG. 13A are used to phase the cardiac waveform in FIG. 13B, and the heart signal is removed from the data sample.

The extracted time series data from the sensors 911 are used to estimate the breathing rate 1008. Lung sounds normally peak at frequencies below 100 Hz (Auscultation of the respiratory system, Sarkar, Malay et al., Annals of thoracic medicine vol. 10, 3 (2015): 158-68, https://www.ncbi.nlm.nih.gov/pmc/articles/PMC4518345/#ref10), with a sharp drop of sound energy occurring between 100 and 200 Hz. Breathing induces oscillations which can be detected by microphones 206-E located inside or at the entrance to ear canal. The breathing cycle is detected by the processing system 106/109 running FFT on a few second long time sample with a moving window at a step much smaller than the breathing time. This step allows the processing system 106/109 to monitor frequency content variable with breathing. The increased power in the frequency range above 20 Hz corresponds to an inhale, while decrease power indicates an exhale. The breathing rate and its characteristics can be also estimated by the processing system 106/109 cross-correlating breathing templates with the time series. The breathing signal is further removed from the time series.

The results of the FFT of such filtered data with remaining brain sound related to brain blood flow and neural oscillations is then spectrally analyzed by the processing system 106/109 using high- and low-pass filters that are applied to restrict the data to a frequency range where brain activity is relatively easy to identify. The brain activity measurement 1009 based on integrating signal in predefined frequency range.

Data Assessment System

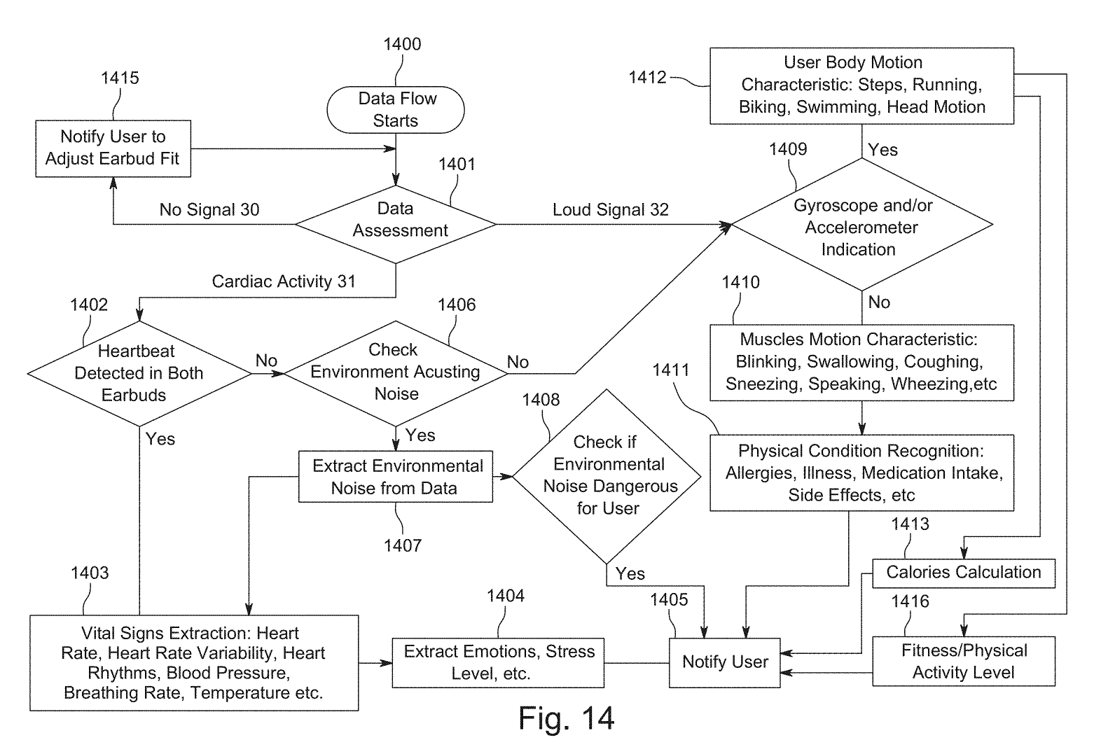

FIG. 14 show a flow chart showing the process performed by the processing system 106/109 to recognize and distinguish cardiac activity, user motion, user facial muscle movement, environmental noise, etc in the data. The biosensor system 50 is activated by a user 10, which starts the data flow 1400 from sensors including internal acoustic sensor 206-E, external/background acoustic sensor 206-B, gyroscope 214, accelerometer 218, magnetometer 222, temperature transducer 225. In the first step, data assessment 1401 is performed by the processing system 106/109 using algorithms based on for example a peak detection of FIG. 13A, and data if flagged as; No Signal 1300, Cardiac Activity 1301, Loud Signal 1302. If the data stream is assessed as No Signal 1300 the system sends notification to a user to adjust right 103 or left 102 earbud position or both to improve the earbud cover 205 seal, which results in acoustic signal amplification in ear canal. If the data stream is assessed as Cardiac Activity 31 by the processing system 106/109, system checks if the heartbeat peaks are detected in right and left earbud in step 1402.

The detection of ventricular contractions simultaneously in right and left ear canal allows the processing system 106/109 to reduce noise level and improve accuracy of the heart rate measurement. The waveform of ventricular contraction is temporarily consistent in both earbuds 102, 103, while other sources of signal may not be correlated, see Loud Signal 1302. Thus, to obtain high accuracy results the system checks if ventricular contractions are detected simultaneously in both earbuds in step 1402. The processing system 106/109 can perform the cardiac activity analysis from a single earbud but with better spurious peak rejection. If the heartbeat is detected in both earbuds, the processing system 106/109 extracts heart rate, heart rate variability, heart rhythm recognition, blood pressure, breathing rate, temperature, etc in step 1403. The extracted values in step 1403 in combination with the previous user data and are used by the processing system 106/109 to extract users emotions, stress level, etc. in step 1404. Following the extraction of parameters in steps 1403 and 1404, the user is notified in step 1045 with the results by the processing system 106/109.

If the data assessment 1401 analysis recognizes cardiac activity 1301, but, not all the heartbeats are detected simultaneously, the processing system 106/109 checks the external/background acoustic sensor 206-B for external level of noise. If external/background acoustic sensor 206-B indicates detection of the acoustic environmental noise 1406 by the processing system 106/109, the data from external/background acoustic sensor 206-B are used to extract environmental acoustic noise from body acoustic signals detected from internal acoustic sensor 206-E. Such extracted environmental noise using external/background acoustic sensor 206-B improves quality of the data produced by the processing system 106/109 and reduces noise level. After extraction of the environmental noise 1407, the data are used by the processing system 106/109 to calculate vital signs 1403 etc.

If the environment acoustic noise 1406 monitored using external/background microphones 206-B indicates significant level of the environmental noise, the processing system 106/109 checks the level and origin of the noise. Next, the processing system 106/109 checks if the detected environmental acoustic noise is dangerous for user 1408. If the level is dangerous, the processing system 106/109 notifies the user 1405.

If the environmental acoustic noise was not detected and/or the data assessment system 1406 recognizes the data as Loud Signal 1302, the data from other sensors 1409 such as gyroscope 214, accelerometer 218, magnetometer 222, etc., are included by the processing system 106/109 to interpret the signal origin. If the data from the auxiliary sensors indicate no user motion, the processing system 106/109 uses a template recognition and machine learning to characterize user muscle motion 1410, which may include blinking, swallowing, coughing, sneezing, speaking, wheezing, chewing, yawing, etc. The data characterization regarding user muscle motion 1410 is used by the processing system 106/109 to detect user physical condition 1411, which may include allergies, illness, medication side effects, etc.

The processing system 106/109 notifies 1405 user if the physical condition 1411 is detected.

If the data from the auxiliary sensors indicate user body motion, the system can use a template recognition or machine learning to characterize user body motion 1412, which may include steps, running, biking, swimming, head motion, jumping, getting up, sitting down, falling, head injury, etc. The data characterization regarding user body motion 1410 can be used to calculate calories burned by the user 1413 and user fitness/physical activity level 1416. The system notifies 1405 user about level of physical activity 1416 and calories burned 1413.

Applications

The portability of the headset and software will allow the processing system 106/109 to take readings throughout the day and night. The processing system 106/109 will push notifications to the user when a previously unidentified biosensor state is detected. This more comprehensive analysis of the user's data will result in biofeedback action suggestions that are better targeted to the user's physical and emotional wellbeing, resulting in greater health improvements.

Biofeedback Parameters: The biosensor data according to an embodiment of the invention enables the processing system 106/109 to provide parameters including but not limited to body temperature, motion characteristics (type, duration, time of occurrence, location, intensity), heart rate, heart rate variability, breathing rate, breathing rate variability, duration and slope of inhale, duration and slope of exhale, cardiac peak characteristic (amplitude, slope, half width at half maximum (HWHM), peak average mean, variance, skewness, kurtosis), relative blood pressure based on for example cardiac peak characteristic, relative blood circulation, filtered brain sound in different frequency ranges, etc.

Biosignal Characteristics: A circadian rhythm is any biological process that displays an endogenous, entrainable oscillation of about 24 hours. Practically every function in the human body has been shown to exhibit circadian rhythmicity. In ambulatory conditions, environmental factors and physical exertion can obscure or enhance the expressed rhythms. The three most commonly monitored and study vital signs are blood pressure (systolic and diastolic), heart rate, and body temperature.

The vital signs exhibit a daily rhythmicity of human vital signs (Rhythmicity of human vital signs, https://www.circadian.org/vital.html). If physical exertion is avoided, the daily rhythm of heart rate is robust even under ambulatory conditions. As a matter of fact, ambulatory conditions enhance the rhythmicity because of the absence of physical activity during sleep time and the presence of activity during the wakefulness hours.

In principle, the heart rate is lower during the sleep hours than during the awake hours. Among the vital signs, body temperature has the most robust rhythm. The rhythm can be disrupted by physical exertion, but it is very reproducible in sedentary users. This implies for example that the concept of fever is dependent on the time of day. Blood pressure is the most irregular measure under ambulatory conditions. Blood pressure falls during sleep, rises at wake-up time, and remains relatively high during the day for approximately 6 hours after waking. Thus, concepts such as hypertension are dependent on the time of day, and a single measurement can be very misleading. The biosensor system 50 that collects user 10 data for an extended period of time can be used to monitor user body clock, known as circadian rhythms.

During sleep physiological demands are reduced. As such, temperature and blood pressure drop. In general, many of physiological functions such as brain wave activity, breathing, and heart rate are variable during waking periods or during REM sleep. However, physiological functions are extremely regular in non-REM sleep. During wakefulness many physiological variables are controlled at levels that are optimal for the body's functioning. Body temperature, blood pressure, and levels of oxygen, carbon dioxide, and glucose in the blood remain constant during wakefulness.

The temperature of our body is controlled by mechanisms such as shivering, sweating, and changing blood flow to the skin, so that body temperature fluctuates minimally around a set level during wakefulness. This process of body temperature controlled is known as thermoregulation. Before falling asleep, bodies begin to lose some heat to the environment, and is believed that this process helps to induce sleep. During sleep, body temperature is reduced by 1 to 2° F. As a result, less energy is used to maintaining body temperature.

During non-REM sleep body temperature is still maintained, however at a reduced level. During REM sleep body temperature falls to its lowest point. Motion such as for example curling up in bed during 10- to 30-minute periods of REM sleep ensures that not too much heat is lost to the environment during this potentially dangerous time without thermoregulation.

Also changes to breathing occur during sleep. In the awaken state, breathing can be irregular because it can be affected by speech, emotions, exercise, posture, and other factors. During transition from wakefulness through the stages of non-REM sleep, breathing rate decreases and becomes very regular. During REM sleep, the breathing pattern becomes much more variable as compared to non-REM sleep and breathing rate increases. As compared to wakefulness, during non-REM sleep there is an overall reduction in heart rate and blood pressure. During REM sleep, however, there is a more pronounced variation in cardiovascular activity, with overall increases in blood pressure and heart rate.

Monitoring of the user's vital signs and biological clock with the biosensor system 50 can be used to help with user's sleep disorders, obesity, mental health disorders, jet lag, and other health problems. It can also improve a user's ability to monitor how their body adjusts to night shift work schedules.

Breathing changes with exercise level. For example, during and immediately after exercise, a healthy adult may have the breathing rate in a range from 35-45 breaths per minute. The breathing rate during extreme exercising can be as high as 60-70 breaths per minute. In addition, the breathing can be increased by certain illnesses, for example fever, asthma, or allergies. Rapid breathing can be also an indication of anxiety and stress, in particular during episodes of anxiety disorder, known as panic attacks during which the affected person hyperventilates. Unusual long-term trends in modification to a person's breath rate can be an indication of chronic anxiety. The breathing rate is also affected by for example everyday stress, excitement, being calm, restfulness, etc.

Too high breathing rate does not provide sufficient time to send oxygen to blood cells. Hyperventilation can cause dizziness, muscle spasms, chest pain, etc. It can also shift normal body temperature. Hyperventilation can also result in difficulty to concentrate, think, or judge situation.

Mental States: Mental states which the biosensors data analysis roughly quantifies and displays to users in the form of a metric may include, but are not limited to, stress, relaxation, concentration, meditation, emotion and/or mood, valence (positiveness/negativeness of mood), arousal (intensity of mood), anxiety, drowsiness, state mental clarity/acute cognitive functioning (i.e. “mental fogginess” vs. “mental clarity”, creativity, reasoning, memory), sleep, sleep quality (for example based on time spent each stage of sleep), sleep phase (REM, non-REM), amount of time asleep at given phase, presence of a seizure, presence of a seizure “prodromal stage” (indicative of an upcoming seizure), presence of stroke or impending stroke, presence of migraine or impending migraine, severity of migraine, heart rate, panic attack or impending panic attack.

Biomarkers for numerous mental and neurological disorders may also be established through biosignal detection and analysis, e.g. using brain infrasound. In addition, multiple disorders may have detectable brain sound footprints with increased brain biodata sample acquisition for a single user and increased user statistics/data. Such disorders may include, but are not limited to, depression, bipolar disorder, generalized anxiety disorder, Alzheimer's disease, schizophrenia, various forms of epilepsy, sleep disorders, panic disorders, ADHD, disorders related to brain oxidation, hypothermia, hyperthermia, hypoxia (using for example measure in changes of the relative blood circulation in the brain), abnormalities in breathing such as hyperventilation.

Added Functionalities: The biosensor system 50 preferably has multiple specially optimized designs depending on their purposes. The head-mounted transducer system 100 may have for example a professional or compact style. The professional style may offer excellent overall performance, a high-quality microphone allowing high quality voice communication (for example: phone calls, voice recording, voice command), and added functionalities. The professional style headset may have a long microphone stalk, which could extend to the middle of the user's cheek or even to their mouth. The compact style may be smaller than the professional designs with the earpiece and microphone for voice communication comprising a single unit. The shape of the compact headsets could be for example rectangular, with a microphone for voice communication located near the top of the user's cheek. Some models may use a head strap to stay in place, while others may clip around the ear. Earphones may go inside the ear and rest in the entrance to the ear canal or at the outer edge of the ear lobe. Some earphones models may have interchangeable speaker cushions that have different shapes allowing users to pick the most comfortable one.

Headsets may be offered for example with mono, stereo, or HD sound. The mono headset models could offer a single earphone and provide sound to one ear. These models could have adequate sound quality for telephone calls and other basic functions. However, users that want to use their physiological activity monitoring headset while they listen to music or play video games could have an option of such headsets with stereo or HD sound quality which may operate at 16 kHz rather than 8 kHz like other stereo headsets.

Physiological activity monitoring headset transducer systems 100 may have a noise cancellation ability by detecting ambient noise and using special software to suppress it, by for example blocking out background noise, which may distract the user or the person they are speaking with over one of the microphones. The noise canceling ability would be also beneficial while the user is listening to music or audiobooks in a crowded place or on public transportation. To ensure effective noise cancellation headset could have more than one microphone. One microphone would be used to detect background noise, while the other to record speaking.

Various embodiments of the invention may include multiple pairing services that would offer users the ability to pair or connect their headset transducer system 100 to more than one Bluetooth-compatible device. For example, a headset with multipoint pairing could easily connects to a smartphone, tablet computer, and laptop simultaneously. The physiological activity monitoring headsets may have a functionality of voice command that may allow users to pair their headset to a device, check battery status, answer calls, reject calls, or even may permit users to access the voice commands included with a smartphone, tablet, or other Bluetooth-enabled devices, to facilitate the use of the headset while cooking, driving, exercising, or working.

Various embodiments of the invention may also include near-field communication (NFC) allowing users to pair a Bluetooth headset with a Bluetooth-enabled device without the need to access settings menus or other tasks. Users could pair NFC-enabled Bluetooth headsets with their favorite devices simply by putting their headset on or near the smartphone, tablet, laptop, or stereo they want to connect to, with encryption technologies keeping communications safe in public networks. The Bluetooth headsets may also use A2DP technology that features dual-channel audio streaming capability. This may allow users to listen to music in full stereo without audio cables. A2DP-enabled headsets would allow users to use certain mobile phone features, such as redial and call waiting, without using their phone directly. A2DP technology embedded into the physiological activity monitoring headset would provide efficient solution for users that use their smartphone to play music or watch videos with ability easy to answer incoming phone calls. Moreover, some embodiments of the biosensor system 50 may use AVRCP technology that use a single interface to control electronic devices that playback audio and video: TVs, high-performance sound systems, etc. AVRCP technology may benefit users that want to use their Bluetooth headset with multiple devices and maintain the ability to control them as well. AVRCP gives users the ability to play, pause, stop, and adjust the volume of their streaming media right from their headset. Various embodiments of the invention may also have an ability to translate foreign languages in real time.

Software

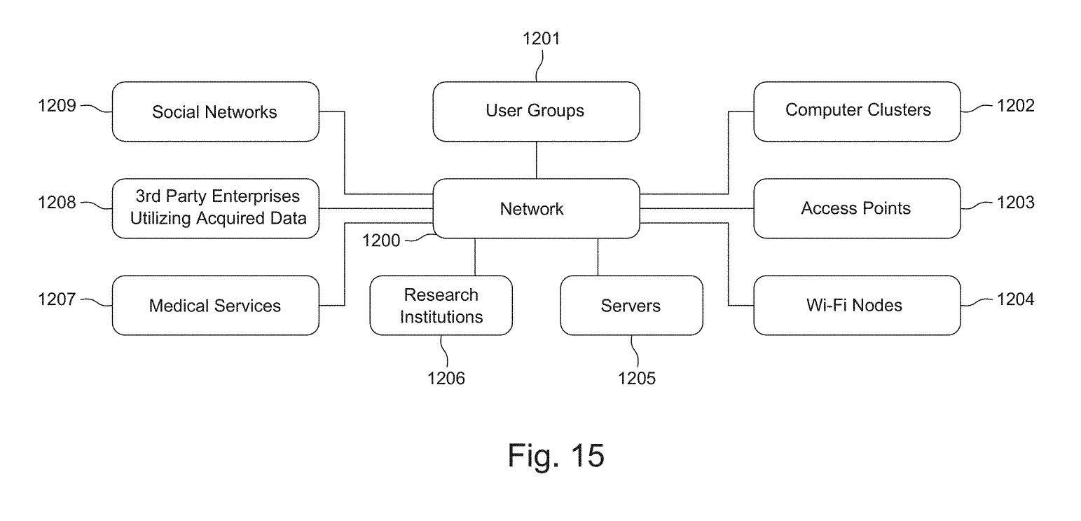

Referring to FIG. 15 there is illustrated a network 1200 supporting communications to and from biosensor systems 50 for various users. Data from these users may be transferred online, e.g. to remote servers, server farms, data centers, computing clouds etc. More complex data analysis may be achieved using online computing resources, i.e. cloud computing and online storage. Each user preferably has the option of sharing data or the results of data analysis using for example social media, social network(s), email, short message services (SMS), blogs, posts, etc. As the network support communication diagram shows, user groups 1201 interface to a telecommunication network 1200 which may include for example long-haul OC-48/OC-192 backbone elements, an OC-48 wide area network (WAN), a Passive Optical Network, and/or a Wireless Link. The network 1200 can be connected to local, regional, and international exchanges and therein to wireless access points (AP) 1203. Wi-Fi nodes 1204 are also connected to the network 1200. The user groups 1201 may be connected to the network 1200 via wired interfaces including, but not limited to, DSL, Dial-Up, DOCSIS, Ethernet, G.hn, ISDN, MoCA, PON, and Power line communication (PLC). The user groups 1201 may communicate to the network 1200 through one or more wireless communications standards such as, for example, IEEE 802.11, IEEE 802.15, IEEE 802.16, IEEE 802.20, UMTS, GSM 850, GSM 900, GSM 1800, GSM 1900, GPRS, ITU-R 5.28, ITU-R 5.150, ITU-R 5.280, and IMT-2000. Electronic devices may support multiple wireless protocols simultaneously, such that for example a user may employ GSM services such as telephony and SMS, Wi-Fi/WiMAX data transmission, VoIP, Internet access etc.

A group of users 1201 may use a variety of electronic devices including for example, laptop computers, portable gaming consoles, tablet computers, smartphones/superphones, cellular telephones/cell phones, portable multimedia players, gaming consoles, and personal computers. Access points 1203, which are also connected to the network 1200, provide, for example, cellular GSM (Global System for Mobile Communications) telephony services as well as 3G, 4G, or 5G evolved services with enhanced data transport support.

Any of the electronic devices may provide and/or support the functionality of the local data acquisition unit 910. Further, servers 1205 which are connected to network 1200. The servers 1205 can receive communications from any electronic devices within user groups 1201. The servers 1205 can also receive communication from other electronic devices connected to the network 1200. The servers 1205 may support the functionality of the local data acquisition unit 910, the local data processing module 920, and as discuss the remote data processing module 930.

External servers connected to network 1200 may include multiple servers, for example servers belonging to research institutions 1206 which may use data and analysis for scientific purposes. The scientific purposes may include but are not limited to developing algorithms to detect and characterize normal and/or abnormal brain and body conditions, studying an impact of the environmental infrasounds on health, characterizing the environmental low frequency signal such as for example from weather, wind turbines, animals, nuclear tests, etc. Also medical services 1207 can be included. The medical services 1207 can use the data for example to track events like episodes of high blood pressure, panic attacks, hyperventilation, or can notify doctors and emergency services in the case of serious events like heart attacks and strokes. Third party enterprises 1208 also may connect to network 1200 for example to determine interest and reaction of users to different products or services, can be used to optimize advertisements that would be more likely of interest to a particular user based on their physiological response. Third party enterprises 1208 may also use the biosensor data to better assess user health, for example fertility and premenstrual syndrome (PMS) by apps such as Clue, respiration and heart rate information by meditation apps such as Breathe.

In addition, network 1200 can allow for connection to social networks 1209 such as or example Facebook, Twitter, LinkedIn, Instagram, Google+, YouTube, Pinterest, Flickr, Reddit, Snapchat, WhatsApp, Quora, Vine, Yelp, and Delicious. A registered user of social networks 1209 may post information related to their physical, emotional states, or information about the environment derived from the biosensor data. Such information may be posted directly for example as a sound, an emoticon, comprehensive data, etc. A user may also customize style and content of information posted on social media and in electronic communications outside the scope of social networking, such as email and SMS. The data sent over the network can be encrypted for example with the TLS protocol for connections over Wi-Fi or for example a SMP protocol for connections over Bluetooth. Other encryption protocols, including proprietary or those developed specifically for this invention may also be used.

The data collected using wearable devices provide a reach and very complex set of information. The complexity of the data often preclude an effective usage of wearable devices because they do not present information in a straightforward and actionable format. Preferably a multi-purpose software bundle is provided that inventors gives an intuitive way of displaying complex biosensor data as app for Android or IOS operating systems, and the software development kit (SDK) to facilitate developers access to biosensor data and algorithms. The SDK represents a collection of libraries (with documentation and examples) designed to simplify the development of biosensor-based applications. The SDK may be optimized for platforms including, but not limited to, iOS, Android, Windows, Blackberry, etc. The SDK have modules that contains biodata-based algorithms for example to extract vital signs, emotional state detection, etc.

The mobile application intended to improve a user's awareness about their emotional and physiological state. The app also allows the monitoring of the infrasound level in the environment. The app uses set of algorithms to extract the user's physiological activity including but not limited to vital signs and uses this information to identify a user's present state. Users can check their physiological state in a real time when they wear the headset with biosensors or can have access to previous data in for example the form of calendar. Actual vital signs and other parameters related to user's body and the environment are displayed when the user is wearing the headset. Moreover, users can see trends showing if the user's current state deviates from normal. The user's normal (baseline) state is estimated using user's long-term data in combination with large set of data from other users and estimations of baseline vitals from the medical field. Users states, trends and correlation with user's actions can be derived using classification algorithms such as for example artificial neural networks, Bayesian linear classifiers, cascading classifiers, conceptual clustering, decision trees, hierarchical classifier, K-nearest neighbor algorithms, K-means algorithms, kernel method, support vector machines, support vector networks, relevance vector machines, relevance vector networks, multilayer perceptron neural networks, neural networks, single layer perceptron models, logistic regression, logistic classifiers, naïve Bayes, linear discriminant analysis, linear regression, signal space projections, hidden Markov models, and random forests. The classification algorithms may be allied to raw, filtered, or pre-processed data from multiple sensors, metadata (e.g. location using Global Positioning System (GPS), date/time information, activity, etc.), vital signs, biomarks, etc.

The present user state can be displayed or vocalized. The app may also vibrate the smartphone/user device 106 to communicate different states or the user's progress. The app can use screen-based push notifications or voice guidance to display or vocalize advice if certain states are detected. For example, if a user's breathing and heart rate will indicate a state of anxiety then the app may suggest breathing exercises. Users may also set their goals to lower their blood pressure or stabilize their breathing. In such situations, the app may suggest appropriate actions. The app will notify the user about their progress and will analyze the user's actions that led to an improvement to or a negative impact on their goals. Users are also able to view their average vitals over time by viewing a calendar or graph, allowing them to keep track of their progress.

The app may interface with a web services provider to provide the user with a more accurate analysis of their past and present mental and physical states. In many instances, more accurate biometrics for a user are too computationally intensive to be calculated on an electronic device and accordingly embodiments of the invention are utilized in conjunction with machine learning algorithms on a cloud-based backend infrastructure. The more data from subjects and individual sessions processed, the more accurate and normative an individual's results will be. Accordingly, the processing tools and established databases can be used to automatically identify biomarkers of physical and psychological states, and as a result, aid diagnosis for users. For example, the app may suggest a user contact a doctor for a particular disorder if the collected and analyzed biodata suggests the possibility of a mental or physical disorder. Cloud based backend processing will allow for the conglomeration of data of different types from multiple users in order to learn how to better calculate the biometrics of interest, screen for disorders, provide lifestyle suggestions, and provide exercise suggestions.

Embodiments of the invention may store data within the remote unit. The apps including the app executing on the user device that use biosensor data may use online storage and analysis of biodata with for example online cloud storage of the cloud computer server system 109. The cloud computing resources can be used for deeper remote analysis, or to share bio-related information on social media. The data stored temporarily on electronic devices can be upload online whenever the electronic device is connected to a network with sufficient battery life or is charging. The app executing on the user device 106 allows storage of temporary data for a longer period of time. The app may prune data when not enough space on the user device 106 is available or there is a connection to upload data online. The data can be removed based on different parameters such as date. The app can also clean storage by removing unused data or by applying space optimization algorithms. The app also allows users to share certain information over social media with friends, doctors, therapists, or a group to, for example, collaborate with a group including other users to enhance and improve their experience of using the biosensor system 50.

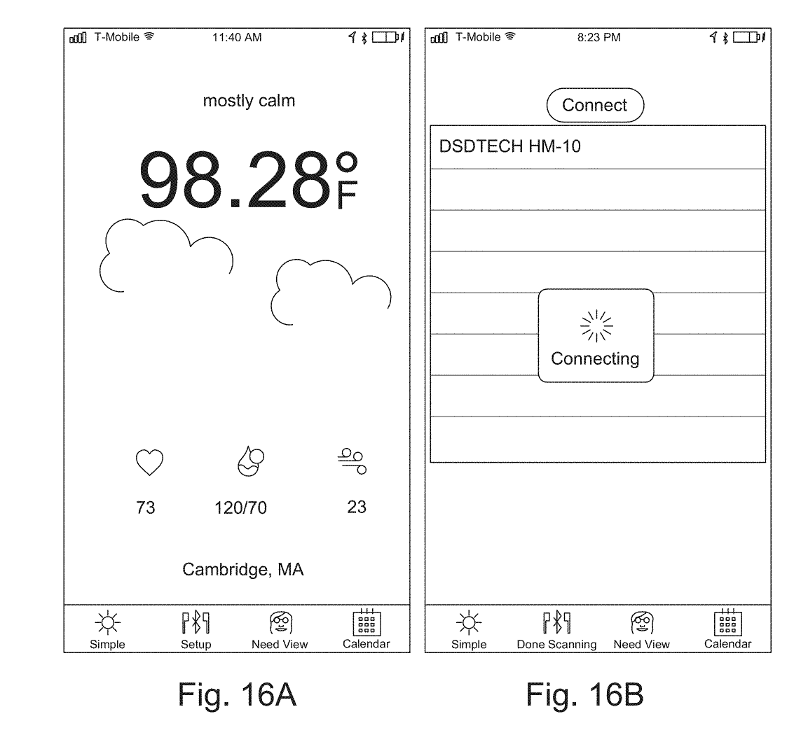

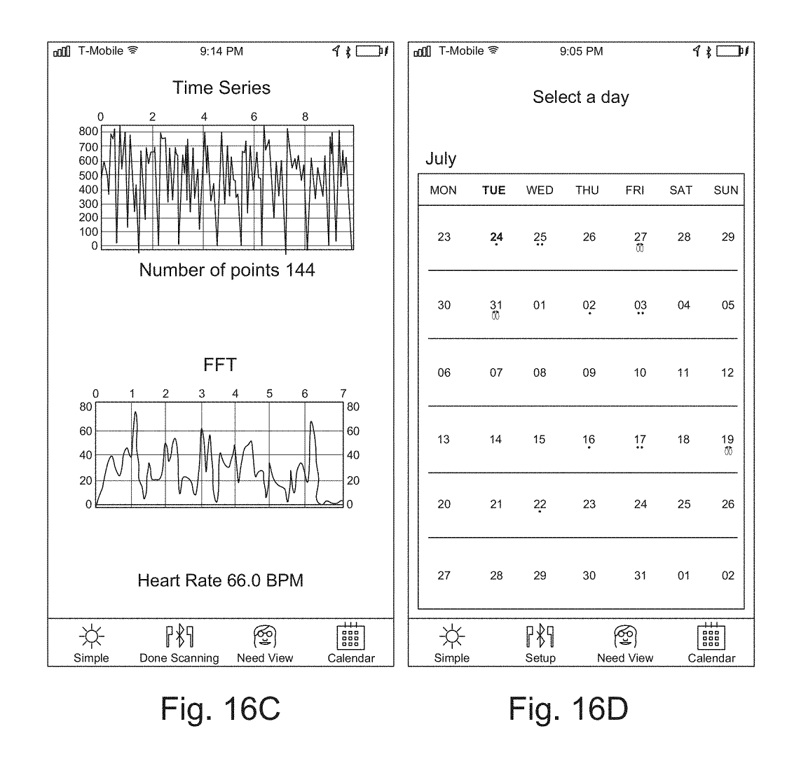

FIGS. 16A-16D shows four exemplary screenshots of the user interface of the app executing on the user device 106. These screenshots are from the touchscreen display of the user device.

FIG. 16A depicts a user's status screen displaying basic vital signs including temperature, heart rate, blood pressure, and breathing rate. The GPS location is also displayed. The background corresponds to user's mental state visualized as and analogized to weather, for example ‘mostly calm’ represented as sky with a few clouds;

FIG. 16B shows a screen of the user interface depicting the Bluetooth connection of the transducer system to their electronic user device 106.

FIG. 16C shows the user interface presenting a more complex data visualization designed for more scientifically literate users. The top of the screen shows the time series from the microphones 206. These time series can be used to check the data quality by for example looking for an amplitude of cardiac cycle. The middle of the screen shows the power spectrum illustrating the frequency content of the signal from microphones 206.

FIG. 16D shows a calendar screen of the user interface of the app executing on the user device 106. Here, the user can check their vital state summary over periods of the biosensor usage.

Diverse applications can be developed that use enhanced interfaces for electronic user devices 106 based on the detection and monitoring of various biosignals. For example, integrating the biosensor data into the feature-rich app development environment for electronic devices in combination with the audio, multimedia, location, and/or movement data can provide a new platform for advanced user-aware interfaces and innovative applications. The applications may include but are not limited to:

Meditation: A smartphone application executing on the user device 106 for an enhanced meditation experience allow users to practice bio-guided meditation anytime and anywhere. Such an application in conjunction with the bio-headset 100 would be a handy tool for improving one's meditation by providing real-time feedback and guidance based on monitored a user's performance estimated based on for example heart rate, temperature, breathing characteristics, or the brain's blood circulation. Numerous types of meditation could be integrated into the system including but not limited to mindfulness meditation, transcendental meditation, alternate nostril breathing, heart rhythm meditation (HRM), Kundalini, guided visualization, Qi Gong, Zazen, Mindfulness, etc. The monitoring of meditation performance combined with information about time and place would also provide users with a better understanding of the impact that the external environment has on their meditation experience. The meditation app would offer a deep insight into user's circadian rhythms and its effects on their meditation. The emotion recognition system based on data from biosensors would allow for the detection of the user's state and suggest an optimal meditation style and would provide feedback. The meditation app would also provide essential data for research purposes.

Brain-Computer Interfaces: The biosensor system 50 allows monitoring of vital signs and mental states such as concentration, emotions, etc., which can be used as a means of direct communication between a user's brain and an electrical device. The transducer system 100 allows for immediate monitoring and analysis of the automatic responses of the body and mind to some external stimuli. The transducer system headset may be used as a non-invasive brain-computer interface allowing for example control of a wide range of robotic devices. The system may enable the user to train over several months to modify the amplitude of their biosignals, or machine-learning approaches can be used to train classifiers embedded in the analysis system in order to minimize the training time.

Gaming: The biosensor system 50 with its ability to monitor vital signs and emotional states could be efficiently implemented by a gaming environment to design more immersive games and provide users with enhanced gaming experiences designed to fit a user's emotional and physical state as determined in real time. For example, challenges and levels of the game could be optimized based on the user's measured mental and physical states.

Sleep: Additional apps executing on the user device 106 can take extensive use of the data from the transducer system 100 to monitor and provide actionable analytics to help users improve the quality of their sleep. The monitored vital signs give insight into the quality of a user's sleep and allow distinguishing different phases of sleep. The information about infrasound in the environment provided by the system would enable the localization of sources of noise that may interfere with the user's sleep. Detection of infrasound in the user's environment and its correlation with the user's sleep quality would provide a unique way to remove otherwise undetectable noises, which in turn would allow users to eliminate such sources of noise and improve the quality of their sleep. The additional information about the user's activity during the day (characteristics and amount of motion, duration of sitting, walking, running, amount of meals) would help to characterization the user's circadian rhythms, which combined with for example machine learning algorithms, would allow the app to detect which actions have a positive or negative impact on a user's sleep quality and quantity. The analysis of the user's vitals and circadian rhythms would enable the app to suggest the best time for a user to fall asleep or wake up. Sleep monitoring earphones could have dedicated designs to ensure comfortability and stability when the user is sleeping. The earbuds designed for sleeping may have also embedded noise canceling solutions.

Fertility Monitoring/menstrual cycle monitoring: The biosensor system 50 also allows for the monitoring of the user's temperature throughout the day. Fertility/menstrual cycle tracking requires a precise measure of a user's temperature at the same time of day, every day. The multi-temporal or all day temperature data collected with the transducer system 100 will allow for tracking of not only one measurement of the user's temperature, but through machine learning and the combination of a single user's data with the collective data of others, can track how a user's temperature will change throughout the day, thus giving a more accurate measure of their fertility. In addition, the conglomerate multi-user/multi-temporal dataset, combined with machine learning algorithms, will allow for the possible detection of any anomalies in a user's fertility menstrual cycle, enabling the possible detection of, but not limited to, infertility, PCOS, hormonal imbalances, etc. The app can send push notifications to a user to let them know where on their fertility menstrual cycle they are, and if any anomalies are detected, the push notifications can include suggestions to contact a physician.

Exercising: The biosensor system 50 allows monitoring vitals when users are exercising providing crucial information about the users' performance. The data provided by the array of sensors in combination with machine learning algorithms may be compiled in the form of a smartphone app that would provide feedback on the best time to exercise optimized based on users' history and a broad set of data. The app executing on the user device 106 may suggest an optimal length and type of exercise to ensure the best sleep quality, brain performance including for example blood circulation, or mindfulness.

iDoctor: The biosensor system 50 also allows real-time detection of a user's body related activity including but not limited to sneezing, coughing, yawning, swallowing, etc. Based on information from a large group of users which has been collected by their respective biosensor systems 50 and analyzed machine learning algorithms executed by the cloud computer server system 109. The cloud computer server system 109 is able to detect and subsequently send push notifications to the user devices 106 of the users about, for example, detected or upcoming cold outbreaks, influenza, sore throat, allergies (including spatial correlation of the source of allergy and comparison with user's history), etc.

The app executing on a user's device 106 may suggest to a user to increase their amount of sleep or exercise or encourage them to see a doctor. The app could monitor how a user's health improves in real time as they take medications, and the app can evaluate if the medication taken has the expected performance and temporal characteristics. The app based on the user's biosensor data may also provide information on detected side effects of the taken medication, or its interaction with other taken medications. The system with embedded machine learning algorithms such as neighborhood-based predictions or a model based reinforced learning would enable the delivery of precision medical care, including patient diagnostic and triage, general patient and medical knowledge, an estimation of patient acuity, and health maps based on global and local crowd-sourced information.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

While this invention has been particularly shown and described with references to preferred embodiments thereof, it will be understood by those skilled in the art that various changes in form and details may be made therein without departing from the scope of the invention encompassed by the appended claims.

What is claimed is:

1. A biosensor system, comprising:
an acoustic sensor for detecting acoustic signals including infrasonic signals from a user via an ear canal; and
a processing system for analyzing the acoustic signals detected by the acoustic sensor.

2. A system as claimed in claim 1, wherein the acoustic signals include infrasounds of 5 Hz and less.

3. A system as claimed in claim 1, further comprising auxiliary sensors for detecting movement of the user.

4. A system as claimed in claim 1, further comprising an auxiliary sensor for detecting body temperature of the user.

5. A system as claimed in claim 4, wherein the acoustic sensor is incorporated into a headset.

6. A system as claimed in claim 1, wherein the headset includes one or more earbuds.

7. A system as claimed in claim 1, further comprising means for occluding the ear canal user to improve an efficiency of the detection of the acoustic signals, wherein the occluding means includes an earbud cover.

8. A system as claimed in claim 1, further comprising acoustic sensors in both ear canals of the user and the processing system using the signals from both sensors to increase an accuracy of a characterization of cardiac activity.

9. A system as claimed in claim 1, wherein the processing system analyzes the acoustic signals to analyze a cardiac cycle and/or respiratory cycle of the user.

10. A method for monitoring a user with a biosensor system, the method comprising:
detecting acoustic signals including infrasonic signal from a user via an ear canal using an acoustic sensor; and
analyzing the acoustic signals detected by the acoustic sensor to monitor the user.

11. A method as claimed in claim 10, wherein the acoustic signals include infrasounds of 5 Hz and less.

12. A method as claimed in claim 10, further comprising detecting movement of the user using auxiliary sensors.

13. A method as claimed in claim 10, further comprising detecting a body temperature of the user.

14. A method as claimed in claim 10, herein the acoustic sensor is incorporated into a headset.

15. A method as claimed in claim 14, wherein the headset includes one or more earbuds.

16. A method as claimed in claim 10, further comprising occluding the ear canal of the user to improve an efficiency of the detection of the acoustic signals.

17. A method as claimed in claim 10, further comprising detecting acoustic signals from both ear canals of the user and using the signals from both canals to increase an accuracy of a characterization of cardiac activity.

18. A method as claimed in claim 10, further comprising analyzing the acoustic signals to track a cardiac cycle and/or a respiratory cycle of the user.

19. An earbud-style head-mounted transducer system, comprising:
an ear canal extension that projects into an ear canal of a user; and
an acoustic sensor in the ear canal extension for detecting acoustic signals from the user.

20. A user device executing an app providing a user interface for a biosensor system on a touchscreen display of the user device, the biosensor system for analyzing infrasonic signals from a user to assess a physical state of the user, the user interface presenting a display that analogizes the state of the user to weather and/or presents the plots of infrasonic signals and/or a calendar screen for accessing past vital state summaries based on the infrasonic signals.

21. A biosensor system and/or its method of operation, comprising:
one or more acoustic sensors for detecting acoustic signals including infrasonic signals from a user; and
a processing system for analyzing the acoustic signals to facilitate one or more of the following:
environmental noise monitoring,
blood pressure monitoring,
blood circulation assessment,
brain activity monitoring,
circadian rhythm monitoring,
characterization of and/or assistance in the remediation of disorders including obesity, mental health, jet lag, and other health problems,
meditation,
sleep monitoring,
fertility monitoring, and/or
menstrual cycle monitoring.

22. A biosensor system and/or method of its operation, comprising:
an acoustic sensor for detecting acoustic signals from a user;
a background acoustic sensor for detecting acoustic signals from an environment of the user; and
a processing system for analyzing the acoustic signals from the user and from the environment.

23. The biosensor system and/or method of claim 22 that characterizes audible sound and/or infrasound in the environment using the background acoustic sensor.

24. The biosensor system and/or method of claim 22 that reduces noise in detected acoustic signals from the user by reference to the detected acoustic signals from the environment and/or information from auxiliary sensors.
